(19) United States
(12) Patent Application Publication
(10) Pub. No.: US 2004/0091498 A1
(43) Pub. Date: May 13, 2004

(54) Defensins: use as antiviral agents

(75) Inventors: Linqi Zhang, Rochelle Park, NJ (US); David D. Ho, Chappaqua, NY (US); Rebecca E. Caffrey, Redwood City, CA (US); Enrique A. Dalmasso, Fremont, CA (US); Jianfeng Mei, Guilford, CT (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Aaron Diamond AIDS Research Center, New York, NY; The Rockefeller University; Ciphergen Biosystems, Inc., Fremont, CA

(21) Appl. No.: 10/452,763

(22) Filed: May 30, 2003

Related U.S. Application Data
(60) Provisional application No. 60/412,414, filed on Sep. 20, 2002.
(60) Provisional application No. 60/384,428, filed on May 31, 2002.
(60) Provisional application No. 60/405,595, filed on Aug. 23, 2002.

(51) Int. Cl. A61K 39/00; A61K 38/17

(52) U.S. Cl. 424/185.1; 514/12

(57) ABSTRACT

The present invention relates to inhibition of viruses, e.g., HIV, using defensins. The invention further relates to methods for identifying and using agents, including small molecule chemical compositions, antibodies, peptides, nucleic acids, antisense nucleic acids, and ribozymes, that increase naturally occurring defensin expression or activity, thereby inhibiting HIV in a cell; as well as to the use of expression profiles and compositions in diagnosis and prophylaxis, and therapy related to HIV infection and related disease states such as AIDS.

### CROSS-REFERENCES TO RELATED APPLICATIONS

The present application is related to U.S. Provisional Patent Application Nos. 60/412,414, filed Sep. 20, 2002, 60/405,595, filed Aug. 23, 2002, and 60/384,428, filed May 31, 2002, the teachings of which are incorporated herein by reference for all purposes.

### BACKGROUND OF THE INVENTION

The present invention relates to inhibition of virus, e.g., HIV, using defensins. The invention further relates to methods for identifying and using agents (such as small organic molecules, antibodies, peptides, nucleic acids (e.g., antisense nucleic acids and ribozymes)) that increase naturally occurring defensin expression or activity, thereby inhibiting HIV in a cell; as well as to the use of expression profiles and compositions in diagnosis, prophylaxis and therapy relating to HIV infection as well as related disease states, such as AIDS.

The Human Immunodeficiency Virus (HIV) infects millions of people globally. Cases are reported from nearly every country amounting to 40 million adults and children living with HIV/AIDS worldwide. In 2001, 5 million people were newly infected with HIV, and there were 3 million adult and child deaths due to HIV/AIDS. A full third of those people living with AIDS are aged 15-24 (World Health Organization, 2001). HIV/AIDS treatments exist, however, the drugs currently used in treatment modalities exhibit numerous side effects, ranging from headaches, diarrhea, fatigue, nausea, tingling sensations, abdominal pain, and reduced appetite to elevated kidney and liver functions. Furthermore, the drugs currently used to treat HIV/AIDS require prolonged treatments that often induce drug resistance and do not result in complete eradication of the virus from the body.

CD8⁺ T lymphocytes are known to play critical roles in the immune responses against HIV-1 infection. While the direct killing of HIV-infected cells by CD8⁺ cytotoxic T lymphocytes (CTL) is important in virus suppression, it has been known for more than 15 years that noncytotoxic, soluble factor(s) secreted by CD8⁺ T lymphocytes are also capable of inhibiting HIV-1 replication in vitro. These nonlytic soluble factors were originally described by Walker and colleagues as the CD8 antiviral factor (“CAF”), a diffusible molecule secreted by stimulated CD8⁺ T cells from certain HIV-1 infected individuals. It has been demonstrated that both CTL-dependent and CAF-associated antiviral activities are correlated with higher CD4⁺ T cell count, improved clinical status and long-term non-progression over longer periods of HIV-1 infection. Unlike CTL, however, the antiviral activity of CAF is non-cytolytic and does not require restriction by major histocompatibility complex class-i molecules or cell-to-cell contact. CAF has been demonstrated to be secreted in greater abundance by stimulated CD8⁺ T lymphocytes from HIV-1 infected persons who are doing well clinically, particularly those characterized as long term non-progressors (“LTNP”) (Levy et al., Immunol. Today, 17:217, 1996, Cao et al., N. Engl. J. Med., 332:201 (1995), Barker et al., Blood, 92:3105 (1998), and Mackewicz et al., J. Clin. Invest., 87:1462 (1991)) In contrast, it is uncommonly secreted by stimulation of CD8⁺ T cells from progressors, i.e. infected patients with evidence of immunodeficiency. In 1999, it was reported that SIV replication in macaques increases dramatically when a monoclonal antibody is used to deplete CD8⁺ cells (X. Jin et al., J. Exp. Med, 189:991 (1999) and J. E. Schmitz et al., Science, 283:857 (1999)). CAF-like activity has been detected in supernatant from stimulated CD8⁺ T cells from SIV-infected rhesus macaques, African green monkeys, HIV-1 infected chimpanzees, and some healthy uninfected humans (Kannagi et al., J. Immunol., 140:2237 (1998), Ennen et al., Proc. Natl. Acad. Sci. USA, 91:7207 (1994), Castro et al., Cell. Immunol., 132:246 (19910, and Hsueh et al., Cell. Immunol., 159:271 (1994)).

Despite tremendous efforts over the past two decades, the identity of CAF has remained elusive (Levy et al., Immunol. Today, 17:217 (1996)). Numerous research groups have attempted to identify CAF, but have been unsuccessful. In 1995, Cocchi et al. showed that stimulated CD8⁺ T lymphocytes can secrete β-chemokines (RANTES, MIP-1α and MIP-1β) that block HIV-1 infection in vitro (Science, 270:1811 (1995)). However, their antiviral activity was observed against macrophage-tropic viral isolates, but not against T-cell-line-tropic strains. This dichotomy was later explained by the discovery that the receptor for β-chemokines, CCR5, also serves as the co-receptor for HIV-1 entry into CD4⁺ T cells (Deng et al., Nature, 381:661 (1996), Dragic et al., Nature, 381:667 (1996), Choe et al., Cell, 85:1135 (1996), Doranz et al., Cell, 85:1149 (1996), and Alkhatib et al., Science, 272:1955 (1996)). Thus, it became apparent that β-chemokines can competitively block so-called R5 viruses that use CCR5 as co-receptor, but not so-called X4 viruses that use an alternate co-receptor, CXCR4 (Feng et al., Science, 272:872 (1996)). Such an antiviral profile clearly distinguished β-chemokines from CAF, which can inhibit both types of HIV-1. Moreover, several studies went on to show that CAF activity could not be eliminated by removing either β-chemokines or Stromal Cell-Derived Factor-1α (“SDF-1α”), the ligand for CXCR4, with specific monoclonal antibodies (Barker et al., Proc. Natl. Acad. Sci. USA, 95:1725 (1998), Moriuchi et al., Proc. Natl. Acad. Sci. USA, 93:15341 (1996), and Lacey et al., Proc. Natl. Acad. Sci. USA, 94:9842 (1997)). Other cytokines have subsequently emerged as possible candidates for CAF, including macrophage-derived chemokine and interleukin-16, but none has stood the test of time (Pal et al., Science, 278:695 (1997), and Baier et al., Nature, 378:563 (1995)).

Thus, CAF is a factor that inhibits HIV replication in vitro and is secreted in greater abundance by stimulated CD8 cells from HIV-infected persons who do not progress to AIDS than those who do progress to AIDS. Despite the discovery of the existence of this factor around 1986 and continued efforts since then by prominent AIDS researchers, no one has been able to determine the identity of CAF. Consequently, the research and medical community has been unable to develop antiviral treatment modalities that rely on the body's own mechanism of virus suppression, CAF secretion.

In view of the foregoing, there exists a need for the identification of CAF and for new therapies that can be used to inhibit HIV replication and/or infection. The present invention fulfills these as well as other needs.

### SUMMARY OF THE INVENTION

It has now been discovered that defensins, i.e., alpha-defensin 1, alpha-defensin 2 and alpha-defensin 3, are responsible for the anti-viral activity of CAF. While it had been known that neutrophils produce alpha-defensins, it has now been discovered that CD8⁺ cells also produce alpha-defensins 1, 2 and 3. It has also been shown that in vitro the alpha-defensins 1, 2, and 3 inhibit HIV replication in CD4⁺ cells. Furthermore, it has been discovered that combinations of alpha-defensin 1 and alpha-defensin 2, or alpha-defensin 1 and alpha-defensin 3, exhibit greater HIV inhibition activity than a single defensin alone.

The present invention is based in part on the discovery that defensins, e.g., alpha-defensins and beta-defensins, have antiviral activity and can be used to treat viral diseases in a patient. In particular, it has also been discovered that defensins, such as alpha-defensins, are responsible for CD8 antiviral activity and in particular, anti-HIV activity. Additionally, it has been found that the combination of two defensins, e.g., alpha-defensin 1 and alpha-defensin 2, has significantly more activity than a single defensin. Accordingly, this invention provides pharmaceutical compositions, prophylactic and therapeutic treatments, diagnostic and prognostic methods and kits, and pharmaceutical screening methods that take advantage of the anti-HIV activity of the defensins.

With the discovery that the defensins are responsible for CAF activity, the present invention provides for the use of defensins in HIV treatment modalities. For example, the administration of defensins to persons either prophylactically or therapeutically can be used to prevent or treat HIV infection. Prophylactic treatments are especially useful for persons at high risk of HIV infection. This invention provides methods of inhibiting HIV replication in a person by administering to the person a pharmaceutically effective amount of a defensin, e.g., an alpha-defensin, and, preferably, at least two defensins, e.g., two alpha-defensins. In certain embodiments, the defensin can be provided in modified form, e.g., as part of a fusion protein comprising a defensin moiety and another polypeptide moiety that enhances the effect of the defensin by, for example, targeting CD4⁺ cells or improving stability or bioavailability in vivo. The present invention also provides pharmaceutical compositions comprising one or more defensins or modified forms thereof in a pharmaceutically acceptable carrier. In another embodiment, methods of inhibiting HIV replication involve administering to a person a composition that activates CD8⁺ cells, which, when stimulated, produce defensins, such as alpha-defensins.

In another embodiment, CD8⁺ cells are expanded ex vivo and monitored for the production of defensins. Thereafter, cells producing defensins are introduced into a person prophylactically or therapeutically.

The present invention also provides treatments involving defensin gene therapy. In one embodiment, a nucleic acid comprising a nucleotide sequence that encodes a defensin or modified form thereof or at least two defensins, is administered to a person. The nucleic acid is thereby incorporated into cells and is transcribed and translated to produce a defensin polypeptide. Again, the nucleotide sequence can encode an alpha-defensin, a beta-defensin, or modified forms thereof, or nucleic acids encoding at least two defensins can be administered. In another embodiment involving the use of gene therapy, cells, such as CD8⁺ cells, are transfected ex vivo with a nucleic acid encoding a defensin, such as an alpha-defensin, so that the transfected cells produce the defensin, and the transfected cells are introduced into the subject.

The methods of inhibiting HIV replication described above as well as herein can be applied to cells cultured in vitro, as well. As such, the methods of the present invention have both in vivo as well as in vitro applications.

Long term non-progressors express significantly greater quantities of defensins, such as alpha-defensins, than those who progress to AIDS. This fact can be used to determine the HIV infection status of a person. That is, by determining whether the amount of defensin in a sample, e.g., serum, urine, blood or other bodily fluid, from a person is above or below a certain amount, one can determine the HIV infection status of that person with respect to whether he or she is likely to progress to AIDS or not. Persons with alpha defensin amounts above a predictive amount (e.g., the average amount present in most AIDS patients) are likely to remain non-progressors, while persons with amounts at or below this amount are likely to progress to AIDS. Treatment regimens can be based on this information, with more aggressive anti-HIV treatments recommended for progressors. In one example, CD8⁺ cells are isolated from a patient, stimulated in vitro, and levels of defensin secretion are measured. Alternatively, one can determine whether the amount of defensin mRNA (or cDNA), e.g., alpha-defensin mRNA, in a sample is above or below a certain amount.

In another embodiment, the present invention provides kits for detecting one or more defensins. Such kits are useful, inter alia, in the prognostic methods of this invention. The kits generally comprise a substrate with means for capturing one or more defensins, such as a microtiter plate or a SELDI mass spectrometry biochip probe derivatized or derivatizable with an anti-defensin antibody or a SELDI mass spectrometry biochip probe with a chromatographic surface that binds the defensins (e.g., WCX or IMAC ProteinChip arrays from Ciphergen Biosystems, Inc., Fremont, Calif.). The kits also can include secondary labeled antibodies for use in a sandwich immunoassay and/or instructions to detect defensin and to correlate the amount of defensin detected with a probable prognosis of HIV infection status and/or AIDS development.

The knowledge that defensins inhibit HIV replication provides direction to discover compounds that modulate defensin activity. Accordingly, this invention provides drug screening assays. In these assays, cells that produce defensins, such as CD8⁺ cells, are exposed to a test compound, and the effect of that compound on production of defensin mRNA, polypeptide, or bioactivity is monitored. Drugs discovered by this method can be administered as pharmaceuticals in the prophylactic or therapeutic treatment of HIV infection.

The present invention also provides methods of detecting polypeptides that bind to a defensin. The polypeptides may be ligands involved in the mechanism of defensin activity. Such methods involve contacting a cell with a defensin. Then, defensin from the cell and polypeptides bound to it are captured on a solid phase and detected. In another embodiment, defensin is bound to the solid phase and the cell contents are contacted with the bound defensin and subsequently detected.

In one aspect, the present invention provides a composition comprising a defensin polypeptide and a pharmaceutically acceptable carrier. In one embodiment, the defensin is an alpha-defensin polypeptide selected from the group consisting of alpha-defensin 1, alpha-defensin 2 and alpha-defensin 3.

In another aspect, the present invention provides a composition comprising a defensin polypeptide and a pharmaceutically acceptable carrier, wherein the composition comprises at least two alpha-defensin polypeptides selected from the group consisting of alpha-defensin 1, alpha-defensin 2 and alpha-defensin 3.

In another aspect, the present invention provides a composition comprising a defensin polypeptide and a pharmaceutically acceptable carrier, wherein the composition comprises at least two alpha-defensins selected from the group consisting of alpha-defensin 1 and alpha-defensin 2.

In another aspect, the present invention provides a composition comprising a defensin polypeptide and a pharmaceutically acceptable carrier, wherein the defensin is an alpha-defensin polypeptide and the composition comprises alpha-defensin 1, alpha-defensin 2, and alpha-defensin 3.

In another aspect, the present invention provides a method of treating HIV infection in a human. The method comprises administering a defensin polypeptide to a human. In one embodiment, the defensin polypeptide is an alpha-defensin polypeptide selected from the group consisting of alpha-defensin 1, alpha-defensin 2, and alpha-defensin 3. In a second embodiment, the method of treating HIV infection further comprises administering a second alpha-defensin polypeptide. The second alpha-defensin polypeptide is selected from the group consisting of alpha-defensin 1, alpha-defensin 2, and alpha-defensin 3.

In another aspect, the present invention provides a method of treating HIV infection in a human, wherein the method comprises administering a therapeutically effective amount of alpha-defensin 1 and alpha-defensin 2 to a human.

In another aspect, the present invention provides a method comprising administering a prophylactic amount of a defensin polypeptide to a person who is at high risk of HIV infection. In one embodiment, the defensin polypeptide is an alpha-defensin polypeptide selected from the group consisting of alpha-defensin 1, alpha-defensin 2, and alpha-defensin 3. In a second embodiment, the method further comprises administering a second alpha-defensin polypeptide to a person who is at high risk of HIV infection. The alpha-defensin polypeptide is selected from the group consisting of alpha-defensin 1, alpha-defensin 2, and alpha-defensin 3. In a third embodiment, the alpha-defensin polypeptide is alpha-defensin 1 and alpha-defensin 2.

In another aspect, the present invention provides a method of inhibiting HIV infection in a CD4⁺ cell in culture. The method comprises the step of contacting the cell with a defensin polypeptide. In one embodiment, the defensin polypeptide is an alpha-defensin polypeptide selected from the group consisting of alpha-defensin 1, alpha-defensin 2, and alpha-defensin 3. In a second embodiment, the method further comprises contacting the cell with a second alpha-defensin polypeptide selected from the group consisting of alpha-defensin 1, alpha-defensin 2, and alpha-defensin 3.

In another aspect, the present invention provides a composition comprising a first nucleic acid encoding a first defensin polypeptide in a pharmaceutically acceptable carrier.

In another aspect, the present invention provides a composition comprising a first nucleic acid encoding a first defensin polypeptide and a second nucleic acid encoding a second defensin polypeptide in a pharmaceutically acceptable carrier. In one embodiment, the first and second defensin polypeptides are selected from the group consisting of alpha-defensin 1, alpha-defensin 2, and alpha-defensin 3. In a second embodiment, the first and second defensin polypeptides are alpha-defensin 1 and alpha-defensin 2.

In another aspect, the present invention provides a method of inhibiting HIV infection in a human comprising transfecting a cell with a nucleic acid comprising a nucleotide sequence encoding a defensin polypeptide.

In another aspect, the present invention provides a method of inhibiting HIV infection in a human comprising transfecting a cell with a nucleic acid comprising a nucleotide sequence encoding a defensin polypeptide, wherein the defensin polypeptide is an alpha-defensin polypeptide selected from the group consisting of alpha-defensin 1, alpha-defensin 2, and alpha-defensin 3. In one embodiment, the cell is a muscle cell. In a second embodiment, the cell is a AC133⁺ or CD34⁺ progenitor cell. In a third embodiment, the cell is a AC133⁺ or CD34⁺ progenitor cell and the transfection is performed ex vivo.

In another aspect, the present invention provides a method of inhibiting HIV infection in a human comprising transfecting a cell with a nucleic acid comprising a nucleotide sequence encoding a defensin polypeptide wherein the nucleic acid comprises an inducible promoter operably linked to the nucleotide sequence encoding the defensin.

In another aspect, the present invention provides a method of determining an individual's HIV status. The method comprises detecting an amount of alpha-defensin in a sample from the individual and correlating the amount with HIV infection status. In one embodiment, the method further comprises correlating the amount of defensin and at least one other indicator selected from the group consisting of CD8⁺ cell count, CD4⁺ cell count, HIV viral load, and total T cell count with HIV infection status. In a second embodiment, the alpha-defensin is detected by ELISA or mass spectrometry, e.g., MALDI or SELDI. In a third embodiment, the alpha-defensin in selected from the group consisting of alpha-defensin 1, alpha-defensin 2, and alpha-defensin 3.

In another aspect, the present invention provides a method of determining an individual's HIV status. The method comprises detecting an amount of alpha-defensin mRNA in a sample from the individual and correlating the amount with HIV infection status. In one embodiment, the alpha-defensin mRNA is selected from the group consisting of alpha-defensin 1 mRNA, alpha-defensin 2 mRNA, and alpha-defensin 3 mRNA.

In another aspect, the present invention provides a method for determining whether a compound modulates alpha-defensin activity in a cell. The method comprises contacting an alpha-defensin producing cell with the compound and determining the functional effect of the compound on alpha-defensin activity. In one embodiment, the cell is selected from a neutrophil, a CD8⁺ cell and an epithelial cell. In a second embodiment, the functional effect is determined by measuring defensin expression levels, cellular proliferation, or HIV replication. In a third embodiment, the compound is a small organic molecule. In a fourth embodiment, the alpha-defensin is selected from the group consisting of alpha-defensin 1, alpha-defensin 2, and alpha-defensin 3.

In another aspect, the present invention provides a method of inhibiting HIV infection in a human comprising administering to the human a compound that activates defensin activity in a cell, e.g., a CD8⁺ cell. In one embodiment, the compound is identified by the method described herein for determining whether a compound modulates alpha-defensin activity in a cell.

In another aspect, the present invention provides a method comprising contacting a cell with an alpha-defensin, contacting an immobilized anti-alpha-defensin antibody with a lysate of the cell, whereby alpha-defensin from the cell is bound to a solid phase, and detecting proteins bound to the immobilized alpha-defensin. In one embodiment, the cell is a CD4⁺ cell, a HeLa cell, a HOS cell or a 2B4 cell. In a second embodiment, the alpha-defensin polypeptide is selected from the group consisting of alpha-defensin 1, alpha-defensin 2, and alpha-defensin 3.

In another aspect, the present invention provides a method comprising contacting a cell with an alpha-defensin, contacting an immobilized anti-alpha-defensin antibody with a lysate of the cell, whereby proteins binding to alpha-defensin are captured, and detecting proteins bound to the immobilized alpha-defensin. In one embodiment, the cell is a CD4⁺ cell, a HeLa cell, or a HOS cell. In a second embodiment, the alpha-defensin polypeptide is selected from the group consisting of alpha-defensin 1, alpha-defensin 2, and alpha-defensin 3.

In another aspect, the present invention provides a kit. The kit comprises a substrate comprising means to bind an antibody; an antibody against an alpha-defensin polypeptide; and instructions to correlate the amount of an alpha-defensin detected in a patient sample with a prognosis of developing AIDS. In one embodiment, the substrate is a mass spectrometer probe. In a second embodiment, the substrate is a microtiter plate. In a third embodiment, the kit further comprises a second antibody specifically directed against alpha-defensin 1, alpha-defensin 2 or alpha-defensin 3. In a fourth embodiment, the second antibody is labeled.

In another aspect, the present invention provides another kit. The kit comprises a substrate comprising means to bind an alpha-defensin polypeptide; and instructions to correlate an amount of an alpha-defensin detected in a patient sample with a prognosis of developing AIDS.

In another aspect, the present invention provides another kit. The kit comprises a first antibody that specifically binds an alpha-defensin and a second antibody that specifically binds alpha-defensin 1, alpha-defensin 2 or alpha-defensin 3.

In another aspect, the present invention provides an alpha-defensin fusion protein comprising a first alpha-defensin polypeptide moiety and a second moiety that binds CD4⁺ cells.

In another aspect, the present invention provides an alpha-defensin fusion protein comprising a first alpha-defensin polypeptide moiety and a second signal peptide moiety (e.g., TPA signal peptide).

In another aspect, the present invention provides a nucleic acid comprising a nucleotide sequence encoding an alpha-defensin fusion protein comprising a first alpha-defensin polypeptide moiety and a second signal peptide moiety (e.g., TPA signal peptide).

In another aspect, the present invention provides an alpha-defensin fusion protein comprising a first alpha-defensin polypeptide moiety and a second moiety that increases stability or bioavailability in vivo.

In another aspect, the present invention provides a pharmaceutical composition comprising a PEGylated alpha-defensin polypeptide and a pharmaceutically acceptable carrier.

In another aspect, the present invention provides an alpha-defensin comprising amino acid substitutions that eliminate proteolytic cleavage sites or amino acid substitutions that improve defensin bioavailability and/or bioactivity.

In another aspect, the present invention provides a method of increasing endogenous alpha-defensin production comprising administering a composition that activates CD8⁺ cells. In one embodiment, the composition comprises an anti-CD3 and other T cell activators.

In another aspect, the present invention provides a method comprising expanding CD8⁺ cells ex vivo, monitoring alpha-defensin production by the CD8⁺ cells, and administering the expanded CD8⁺ cells to an HLA-matched person.

In another aspect, the present invention provides a method comprising administering a vaccine and an alpha-defensin polypeptide or nucleic acid encoding alpha-defensin to a person.

In another aspect, the present invention provides a composition comprising a vaccine and an alpha-defensin polypeptide or a nucleic acid encoding an alpha-defensin.

In another aspect, the present invention provides a composition comprising an alpha-defensin and a second therapeutic agent or agents. In one embodiment, the second therapeutic agent is used to prevent or treat HIV infection. In another embodiment, the second therapeutic is used to treat an opportunistic infection associated with HIV infection. In another embodiment, the second therapeutic agent is a protease inhibitor, a nonnucleoside reverse transcriptase inhibitor, a nucleoside reverse transcriptase inhibitor, a fusion inhibitor, an antiretroviral nucleoside, an entry inhibitor, or any other anti-viral agent effective to inhibit or treat HIV infection. In another embodiment, the second therapeutic agent is selected from the group consisting of zidovudine, didanosine, stavudine, interferon, lamivudine, adefovir, nevirapine, delaviridine, loviride, saquinavir, indinavir, AZT, T20, T22 and T2. In another embodiment, the second therapeutic agent is an antibiotic or acyclovir. In another embodiment, the alpha-defensin is selected from the group consisting of alpha-defensin 1, alpha-defensin 2, and alpha-defensin 3. In another embodiment, the composition comprises two or more alpha-defensins. In another embodiment, the alpha-defensin is an alpha-defensin 1, 2 or 3 protein, a nucleotide sequence encoding such an alpha-defensin protein, a fusion protein, or a nucleotide sequence encoding such fusion protein.

In another aspect, the present invention provides methods of treating or preventing HIV infection in a human comprising administering a defensin polypeptide to the human in combination with a second therapeutic agent(s). In one embodiment, the second therapeutic agent is used to prevent or treat HIV infection. In another embodiment, the second therapeutic is used to treat an opportunistic infection associated with HIV infection. In another embodiment, the second therapeutic agent is a protease inhibitor, a nonnucleoside reverse transcriptase inhibitor, a nucleoside reverse transcriptase inhibitor, a fusion inhibitor, an antiretroviral nucleoside, an entry inhibitor, or any other anti-viral agent effective to inhibit or treat HIV infection. In another embodiment, the second therapeutic agent is selected from the group consisting of zidovudine, didanosine, stavudine, interferon, lamivudine, adefovir, nevirapine, delaviridine, loviride, saquinavir, indinavir, AZT, T20, T22 and T2. In another embodiment, the second therapeutic agent is an antibiotic or acyclovir. In another embodiment, the alpha-defensin is selected from the group consisting of alpha-defensin 1, alpha-defensin 2, and alpha-defensin 3. In another embodiment, the composition comprises two or more alpha-defensins. In another embodiment, the alpha-defensin is a defensin protein, a nucleotide sequence encoding a defensin protein, a fusion protein, or a nucleotide sequence encoding a fusion protein.

In another aspect, the present invention provides methods of inhibiting HIV infection in a CD4⁺ culture comprising the step of contacting the cell with a defensin polypeptide and a second therapeutic agent or a combination of other therapeutic agents. In one embodiment, the therapeutic agent or agents are used to treat or prevent HIV infection. In a second embodiment, the therapeutic agent is selected from the group consisting of a protease inhibitor, a nonnucleoside reverse transcriptase inhibitor, a fusion inhibitor, a nucleoside reverse transcriptase inhibitor, an antiretroviral nucleoside, an entry inhibitor, and any other anti-viral agent effective to inhibit or treat HIV infection. In a third embodiment, a therapeutic agent is selected from the group consisting of zidovudine, didanosine, stavudine, interferon, lamivudine, adefovir, nevirapine, delaviridine, loviride, saquinavir, indinavir, AZT, T20, T22 and T2.

### BRIEF DESCRIPTION OF THE DRAWINGS

FIG. 1: Representative protein mass spectra of culture supernatants from stimulated and unstimulated CD8 T cells from two LTNP, one normal individual, and one progressor. Protein peaks that are up-regulated after stimulation are highlighted and their masses are indicated.

FIG. 2: 2(a) Identification of protein peaks with molecular masses of 3,371.9 Da, 3,442.5 Da, and 3,486.5 Da as human α-defensins-2, -1 and -3, using beads coated with an anti-α-defensins-1,2,3 antibody. 2(b) Protein sequencing of a unique 1060.50 Da peptide fragment after trypsin digestion (upper right), using tandem mass spectrometry. Unique peaks from collision-induced dissociation of 1060.50 Da parent ion are indicated. Protein Prospector MS-Tag search of NCBI and SwissProt database showed that the peptide fragment, YGTCIYQGR (highlighted in the upper left), from α-defensins-1, -2, and -3 was the best match for the 1060.50 Da parent ion, with a Probability Based Mowse Score of 49 (Mascot software from Matrix Science). The next closest match (β-galactosidase precursor, 76,091 Da) had a score of only 17. In such analyses, a Mowse score over 38 is regarded as positive identification or extreme homology.

FIG. 3: (a) Antiviral activity against a panel of X4 and R5 HIV-1's before (solid) and after (hatched) depletion of α-defensins-1, -2 and -3 from culture supernatants of LTNP-3 and LTNP-5. The names of the viral isolates are as indicated, and the HIV-1 genotypes are shown in parentheses. The error bars indicate the standard deviation from the mean of two independent experiments. 3(b) Antiviral activity of culture supernatants from stimulated CD8 T cells from LTNP-3 and LTNP-5 in the presence of increasing amounts of antibodies against α-defensins-1, -2 and -3 (left panels) or in combination with that against β-chemokines (right panels).

FIG. 4: Anti-HIV-1 activity of commercially available α-defensins-1 and -2 peptides (left panel) and purified α-defensins-1, -2 and -3 (right panel). The unconnected symbols at the lower right corner of each panel denote the antiviral activity of the highest concentration of α-defensins when an anti-α-defensin monoclonal antibody (25 μg/ml) is also added.

FIG. 5: Immunofluorescence staining of α-defensins-1, -2, and -3 in human neutrophils as well as in unstimulated and stimulated CD8 T lymphocytes. The procedure was carried out as described (33) such that α-defensins stain in green, CD8 proteins in red, and nuclei in blue.

FIG. 6: Changes in molecular mass/charge before and after reduction with dithiothreitol (DTT).

FIG. 7: Mass spectrometry profiles of commercial preparations of α-defensin-1 and -2 as compared to that found in the supernatant of stimulated CD8 T cells from Normal-2.

FIG. 8: α-defensins-1, -2, and -3 purified from neutrophils of a normal person is virtually indistinguishable from those released from stimulated CD8 T cells.

FIG. 9: Number of αβ CD8 T cells expressing α-defensins on day 0, 1, and 2 after stimulation. The Y-axis denotes the number of events detected.

FIG. 10: Alignment of of known alpha-defensins from human, mice, rat, guinea pig and rabbit.

### DETAILED DESCRIPTION OF THE INVENTION

### A. General Overview

The present invention provides new compositions, assays, kits and methods of preventing, treating, diagnosing or predicting viral infection, e.g., HIV infection, killing virally infected cells, e.g., HIV infected cells, and generally, inhibiting viral, preferably HIV, replication. The present invention is based, in part, on the surprising discovery that naturally occurring defensins are present in increased levels in individuals infected with HIV whose disease state has not progressed to AIDS despite 10 years of infection as compared to individuals infected with HIV who have AIDS, and that naturally occurring defensins are preventing HIV from replicating in those individuals who have not acquired full-blown AIDS despite long-term infection. Accordingly, this invention is based, in part, on the surprising discovery that defensins or fragments thereof, when increased in an individual, whether by exogenous administration or by increased endogenous production, inhibit HIV infection, kill HIV infected cells and/or prevent HIV infection in the individual.

The present invention provides methods of increasing levels of exogenous defensins in individuals by administering therapeutic compounds, e.g., defensin proteins (synthetic, recombinant or naturally occurring), defensin nucleic acids, and pharmaceutical compositions comprising one or more defensins, to individuals infected with HIV or at risk of HIV infection.

The present invention also provides methods of inhibiting HIV infection or replication by providing methods of increasing endogenous defensin activity. For example, the present invention provides assays for the identification of modulators of defensin activity. Such modulators of defensin activity are useful for inhibiting HIV infection and replication by increasing levels of endogenous defensin in the body.

The present invention also provides methods of determining the prognosis of individuals infected with HIV. By detecting levels of endogenous defensin in an individual, e.g., defensin levels in neutrophils, a determination can be made whether HIV infection in an individual will progress to AIDS. Such a determination can aid in choices for drug administration, treatment strategies, and duration of treatment.

The present invention also provides compositions comprising a defensin polypeptide and a pharmaceutically acceptable carrier for delivery to individuals infected with HIV or at risk of HIV infection. Such compositions can be used to treat HIV infection in an individual.

The present invention also provides kits for diagnosing and prognosing HIV infection and AIDS development in an individual.

### B. Definitions

The phrase “disorder associated with HIV infection” or “disease associated with HIV infection” herein refers to a disease state which is marked by HIV infection. Such disorders associated with HIV infection include, but are not limited to, AIDS; Kaposi's sarcoma; opportunistic infections such as those caused by Pneumocystis carinii and Mycobacterium tuberculosis; oral lesions, including thrush, hairy leukoplakia, and aphthous ulcers; generalized lymphadenopathy; shingles; thrombocytopenia; aseptic meningitis; neurologic disease such as toxoplasmosis, cryptococcosis, CMV infection, primary CNS lymphoma, and HIV-associated dementia; peripheral neuropathies, seizures; and myopathy.

The phrase “long term non-progressors” refers to individuals who have been infected with HIV for approximately 10 years or longer, who are characterized by normal and stable levels of CD4⁺ T cells, and who have not been treated with antiretroviral therapy.

A person is a “high risk” of HIV infection if that person belongs to a group whose risk of HIV infection is higher than the risk of the population as a whole.

The phrase “functional effects” in the context of assays for testing compounds that modulate activity of a defensin polypeptide includes the determination of a parameter that is indirectly or directly under the influence of a defensin, e.g., a phenotypic or chemical effect, such as the ability to increase or decrease HIV infection, HIV replication, display of HIV markers by HIV infected cells, or cellular (e.g., lymphocyte, preferably CD4⁺ lymphocyte) proliferation, apoptosis; or e.g., a physical effect such as ligand binding or inhibition of ligand binding. A functional effect therefore includes ligand binding activity, the ability of cells to proliferate, ability of HIV to infect a cell and express viral proteins, apoptosis, and enzyme activity. “Functional effects” include in vitro, in vivo, and ex vivo activities.

The phrase “determining the functional effect” refers to assaying for a compound that increases or decreases a parameter that is indirectly or directly under the influence of a defensin protein, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape); chromatographic; or solubility properties for the protein; measuring inducible markers or transcriptional activation of the protein; measuring binding activity or binding assays, e.g., binding to antibodies; measuring changes in ligand or substrate binding activity; measuring cellular proliferation; measuring apoptosis; measuring viral protein expression; measuring viral replication; measuring viral titer or viral RNA copies in serum; measurement of changes in protein levels for defensin or defensin-associated sequences; measurement of RNA stability; identification of downstream or reporter gene expression (CAT, luciferase, β-gal, GFP and the like), e.g., via chemiluminescence, fluorescence, colorimetric reactions, antibody binding, and inducible markers.

“Inhibitors,” “activators,” and “modulators” of defensin polynucleotide and polypeptide sequences are used to refer to activating, inhibitory, or modulating molecules identified using in vitro and in vivo assays of defensin polynucleotide and polypeptide sequences. Inhibitors are compounds that, e.g., bind to, partially or totally block activity, decrease, prevent, delay activation, inactivate, desensitize, or down regulate the activity or expression of defensin proteins, e.g., antagonists. “Activators” are compounds that increase, open, activate, facilitate, enhance activation, sensitize, agonize, or up regulate defensin protein activity or defensin expression, e.g., agonists. Inhibitors, activators, or modulators also include genetically modified versions of defensin proteins, e.g., versions with altered activity, as well as naturally occurring and synthetic ligands, antagonists, agonists, antibodies, peptides, cyclic peptides, nucleic acids, antisense molecules, ribozymes, small chemical molecules and the like. Such assays for inhibitors and activators include, e.g., expressing defensin protein in vitro, in cells, or cell membranes, applying putative modulator compounds, and then determining the functional effects on activity, as described above.

Samples or assays comprising defensin proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of defensin is achieved when the activity value relative to the control is about 80%, preferably 50%, more preferably 25-0%. Activation of defensin is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator,” as used herein, describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc. Such molecules can be tested for the capacity to directly or indirectly modulate viral infection, e.g., HIV infection. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons. A “small organic molecule” as described herein is distinct from a peptide.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding a defensin described herein or amino acid sequence of a defensin described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Natl. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res., 25:3389-3402 (1977) and Altschul et al., J. Mol. Biol., 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells may express genes that are not found within the native (non-recombinant) form of the cell or express native genes that are otherwise abnormally expressed, under expressed or not expressed at all. Recombinant cells may also express native genes whose expression has been turned on by the insertion of regulatory DNA sequences, such as promoters or enhancers.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash inf 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature, 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today, 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology, 10:779-783 (1992); Lonberg et al., Nature, 368:856-859 (1994); Morrison, Nature, 368:812-13 (1994); Fishwild et al., Nature Biotechnology, 14:845-51 (1996); Neuberger, Nature Biotechnology, 14:826 (1996); and Lonberg & Huszar, Intern. Rev. Immunol., 13:65-93 (1995)). Alternatively, phage display technology can be used to identify antibodies and heteromeric Fab fragments that specifically bind to selected antigens (see, e.g., McCafferty et al., Nature, 348:552-554 (1990); Marks et al., Biotechnology, 10:779-783 (1992)). Antibodies can also be made bispecific, i.e., able to recognize two different antigens (see, e.g., WO 93/08829, Traunecker et al., EMBO J., 10:3655-3659 (1991); and Suresh et al., Methods in Enzymology, 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature, 321:522-525 (1986); Riechmann et al., Nature, 332:323-327 (1988); Verhoeyen et al., Science, 239:1534-1536 (1988) and Presta, Curr. Op. Struct. Biol., 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a defensin protein, polymorphic variants, alleles, orthologs, and conservatively modified variants, or splice variants, or portions thereof, can be selected to obtain only those polyclonal antibodies that are specifically immunoreactive with defensin proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces the desired effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)). In the case of a therapeutically effective amount of a defensin for use as an anti-HIV agent, the therapeutically effective amount will be an amount necessary to achieve any indicia of success in the treatment of HIV infection or AIDS in an individual, including any objective or subjective critera such as HIV viral inhibition, diminishing of symptoms associated with HIV infection and AIDS, or improvement of a patent's physical or mental well-being. For example, for some patients, a therapeutically effective dose will be from 1 microgram per kg of body weight to 1 gram per kg of body weight.

### C. Defensins: Preparation and Purification

### 1. The Defensins

Defensins are well known in the art as anti-bacterial or anti-fungal agents (see, U.S. Pat. No. 5,242,902, which issued to Murphy et al). Defensins have been found and characterized in many animals, including humans, guinea pig, rat, rabbit, macaques, and mice, as well as in plants and insects.

For the purposes of the present invention, there are two structural classes of mammalian defensins, α and β. The term “defensin,” as used in this application, includes both α and β defensins, and in particular α-defensins-1, 2, and 3. The genes encoding the human defensins are thought to be located on a single chromosomal region, 8p21-23 (Kaiser et al., Journal of Leukocyte Biology, 68:779-784 (2000)). Structurally, defensins are cationic molecules with spatially separated hydrophobic and charged regions. The known defensins all share a β sheet structure and 6 or 8 cysteine residues that form intramolecular cysteine disulfide bonds.

As used herein, the term “α-defensin” refers to a polypeptide whose biological activity is characterized as having anti-HIV activity as determined using an HIV virus inhibition assay, such as the virus inhibition assay described herein. The alpha-defensins are generally less than 100 amino acids longs, usually about 25-35 amino acids long, characterized by six cysteine residues in a motif that is conserved between species. (The conserved motif is apparent from SEQ ID NO:3 to SEQ ID NO:8 herein; see also, Liu and Ganz, Genomics, 43:316-320 (1997); and U.S. Pat. No. 4,705,777 (Lehrer et al.)). The alpha-defensins also have a net positive charge at physiological pH, generally attributable to positively charged arginines. Accordingly, the term α-defensin encompasses cationic proteins having both high cysteine and high arginine content. In some embodiments, the α-defensins are characterized having six cysteines and two to four arginines, which are substantially conserved. The cysteines and arginines are dispersed throughout the oligopeptide, so that the cysteines provide for the opportunity for extensive crosslinking, intramolecularly and intermolecularly, covalently and noncovalently, and the arginines provide for positive charges throughout the molecule at a wide range of pHs, so as to be highly cationic. For example, in some embodiments, the α-defensins will contain 3 disulfide bonds, formed by the cysteine residues. The α-defensins of the present invention may also form a secondary structure comprising a β hairpin conformation, such as a β sheet, e.g., a triple-stranded antiparallel β sheet (Mandal et al., J. Peptide Research, 59:95-104 (2002)). The term “alpha-defensin” embraces naturally occurring alpha-defensins, i.e., polypeptides isolated from natural sources; synthetic alpha-defensins, i.e., polypeptides chemically synthesized or produced by recombinant DNA techniques and having the amino acid sequence of a naturally occurring α-defensin; and alpha-defensin analogs, i.e., polypeptides having the biological activity of an α-defensin and having an amino acid sequence that is not naturally occurring. The term also embraces pre-proprotein forms of alpha-defensins that are processed intracellularly into a biologically active form.

α-defensins are expressed in various sites throughout the body. However, α-defensins 1-4 are predominantly found in the polymorphonuclear neutrophils, whereas the α-defensins 5-6 are highly expressed in the secretory granules of Paneth cells in the small intestine. Human alpha-defensins 1-3 have now been identified as being secreted by CD8⁺ T cells. Three of the defensins, i.e., α-defensins-1-3, differ by only one amino acid at the N-terminus (Linzmeier et al., FEBS LETT., 32](2-3):267-273 (1993); Palfree et al., Mol. Endocrin., 7(2):199-205 (1993); Mallow et al., J. Biol. Chem., 271(8):4038-4045; (1996); Mars et al., J. Biol. Chem., 270(51):30371-30376; and Quayle et al., Am. J. Pathol., 152(5): 1247-1258).

In certain embodiments, α-defensins, as defined above, include polypeptides, pre-proproteins, processed proteins, polymorphic variants, alleles, and mutants that have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 15, 20, 25, 30 or more amino acids, to an amino acid sequence encoded by a human α-defensin nucleic acid as described herein, e.g., α-defensins 1-6, preferably α-defensins 1-3.

In other embodiments, α-defensins, as defined above, include polypeptides, pre-proproteins, processed proteins, polymorphic variants, alleles, mutants and interspecies homologs that have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 15, 20, 25, 30 or more amino acids, to an amino acid sequence encoded by an α-defensin nucleic acid from humans and other animals as described herein, e.g., rabbit defensins 1-5, rat defensins 1-4, and guinea pig defensin.

In other embodiments, α-defensins, as defined above, include polypeptides, pre-proproteins, processed proteins, polymorphic variants, alleles, mutants and interspecies homologs that have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 15, 20, 25, 30 or more amino acids, to an amino acid sequence encoded by an α-defensin nucleic acid highly expressed in Paneth cells, e.g., human α-defensins 5-6, mouse α-defensins 1,2, and 1α and rabbit α-defensin-6.

Alternatively, α-defensins, as defined above, include cationic oligopeptides having no more than 35 amino acids and having a sequence of the formula: Z₀₋₁-(aa¹)ₐ-(aa²)_{b}-cys-aa⁴-cys-arg-aa⁷-aa⁸-aa⁹-cys-aa¹¹-aa¹²-aa¹³-glu-arg-aa¹⁶-aa¹⁷-gly-aa¹⁹-cys-aa²¹-aa²²-aa²³-gly-aa²⁵-aa²⁶aa²⁷-aa²⁸-aa²⁹-cys-cys-(aa³²)_{c}-w (SEQ ID NO:1), wherein: Z, if present, is chemically coupled to the terminal amino group and can be an acyl group of from one to six carbon atoms having from zero to one amino substituent, alkyl of from one to three carbon atoms or a protective group; a, b and c are independently either 0 or 1; the superscripts to the aa which defines amino acid refers to the amino acid number in the polypeptide except for aa⁹ where it is intended that two amino acids are present and then all subsequent numbers are increased by one, as well as the numbers in the subsequent definitions; amino acids 1, 7, 8, 11, 13, 21, 23, 25, 26 and 28 are aliphatic amino acids; amino acids 2, 4, 9, 12, 16, 17, 19, 22, 27, 29 and 32 are either aliphatic amino acids or aromatic amino acids; and w is the terminal hydroxyl, amino or a peptide of from one to six amino acids having a basic amino acid at the N terminus. In some embodiments, aa¹ is val or gly; aa² is val, ile, arg ser, phe, ala or asp; aa⁴ is ala, val, thr or tyr; aa⁷ is arg, lys, gly or ile; aa⁸ is ala, arg, gln, phe or pro; aa⁹ is two leu, phe, ser or ala; aa¹¹ is leu, pro, ser, gly or ile; aa¹² is pro, asn, lys, phe, ser or ala; aa¹³ is arg, leu, ser or gly; aa¹⁶ is arg, phe or ala; aa¹⁷ is ala, ser, ile or tyr; aa¹⁹ is phe, tyr, asp, ser or thr;aa²¹ is arg, lys, thr or ile; aa²² is ile, val or tyr; aa²³ is arg, asn or gln; aa²⁵ is arg, ala or val; aa²⁶ is ile, leu or arg; aa²⁷ is his, val, phe or trp; aa²⁸ is pro, tyr, ala or thr; aa²⁹ is leu, arg or phe; aa³² is arg, ser, pro or trip; and w is zero to two arg. In other embodiments, the substituents, such as aa¹, aa², and aa⁴, are secreted such that they have other than the naturally occurring sequences and are referred to herein as analogs.

Additionally, α-defensins, as defined above, include cationic oligopeptides having no more than 35 amino acids having a sequence of the formula: Z′₀₋₂-val-aa²-cys-aa⁴-cys-arg-arg-aa⁸-aa⁹-cys-aa¹¹-aa¹²-aa¹³-glu-arg-arg-aa¹⁷-gly-aa¹⁹cys-arg-aa²²-arg-gly-arg-aa²⁶-his-aa²⁸-leu-cys-cys-arg-(arg)₀₋₁ (SEQ ID NO:2), and wherein Z′ is methyl, acetyl or an amino acid; amino acids 2, 4, 8, 9, 11, 17, and 22 are neutral amino acids; amino acids 12 and 28 are heterocyclic amino acids or neutral amino acids; amino acid 19 is an aromatic amino acid or hydroxy substituted aliphatic amino acid; amino acids 13 and 26 are aliphatic amino acids or basic amino acids; or having a sequence of the formula: Z′₀₋₂-val-aa²-cys-thr-cys-arg-aa⁷-phe-aa⁹-cys-gly-aa¹²-gly-glu-arg-ala-aa¹⁷-gly-aa¹⁹-cys-thr-aa²²-asn-gly-val-arg-his-aa²⁸-leu-cys-cys-arg-(arg)₀₋₁ and wherein: aa² and aa¹² are phe or ser; aa⁷is arg or gly; aa⁹ is a hydroxy substituted or unsubstituted aliphatic amino acid; aa¹⁷, aa¹⁹ and aa²⁸ are hydroxy substituted amino acids; aa²² is an aliphatic amino acid of from 5 to 6 carbon atoms; or having a sequence of the formula ala-cys-tyr-cys-arg-ile-pro-ala-cys-ile-ala-asp-gly-glu-arg-arg-tyr-gly-thr-cys-ile-tyr-gln-gly-arg-leu-trp-ala-phe-cys-cys, wherein ala, and asp indicate either no amino acid or one of the indicated amino acids. In other embodiments, the substituents, such as aa², aa⁷, and aa⁹, are secreted such that they have other than the naturally occurring sequences and are referred to herein as analogs.

Alpha-defensins have been found in human, rabbit, guinea pig, rat, macaque, and hamster neutrophils, in rabbit alveolar macrophages, and human and rodent small-intestinal Paneth cells (Daher et al., Proc. Natl Acad. Sci. USA, 85:7327-7331, Lehrer et al., Infect. Immun., 11:1226-1234, Selsted et al., Infect. Immun., 55:2181-2186, Eisenhauer et al., Infect. Immun., 57:2021-2027, Tang et al., Infect. Immun., 67:6139-6144, Mak et al., Infect. Immun., 64:4444-4449, Ganz et al., J. Immunol., 143:1358-1365, Mallow et al., J. Biol. Chem., 271:4038-4045, Ouellette et al., J. Cell. Biol., 108:1687-1695, and Qu et al., Infect. Immun., 64:5161-5165).

As used herein, the term “human alpha-defensin” refers to a polypeptide having one of the following amino acid sequences, allelic variants thereof, and pre-proproteins thereof. The sequences for human alpha-defensins 1-6 are as follows; Alpha-defensin 1 (HNP1): ACYCRIPACIAGERRYGTCIYQGRLWAFCC (SEQ ID NO:3); Alpha-defensin 2 (HNP2): CYCRIPACIAGERRYGTCIYQGRLWAFCC (SEQ ID NO:4); Alpha-defensin 3 (HNP3): DCYCRIPACIAGERRYGTCIYQGRLWAFCC (SEQ ID NO:5), Alpha-defensin 4 (HNP4): VCSCRLVFCRRTELRVGNCLIGGVSFTYCCTRV (SEQ ID NO:6), Alpha-defensin 5 (HD5): ARATCYCRTGRCATRESLSGVCEISGRLY RLCCR (SEQ ID NO:7), and Alpha-defensin 6 (HD6): TRAFTCHCRRSCYSTEYSYGTCT VM GINHRFCCL (SEQ ID NO:8). Alpha-defensins are described in U.S. Pat. No. 4,705,777 (Lehrer et al.).

As used herein the term “rabbit alpha-defensin” refers to a polypeptide having one of the following amino acid sequences, allelic variants thereof, and pre-proproteins thereof. The sequence for rabbit alpha-defensins 1-6 are as follows: Alpha-defensin 1 (NP1): VVCACRRALCLPRERRAGFCRIRGRIHPLCCRR (SEQ ID NO:9), Alpha-defensin 2 (NP2): VVCACRRALCLPLERRAGFCRIRGRIHPLCCRR (SEQ ID NO:10), Alpha-defensin 3a (NP3a): GICACRRRFCPNSERFSGYCRVNGARYVRCCSRR (SEQ ID NO:11), Alpha-defensin 3b (NP3b): GRCVCRKQLLCSYRERRIGDCKIRGVRF PFCCPR (SEQ ID NO:12), Alpha-defensin 4 (NP4): VSCTCRRFSCGFGERASGSCTV NGVRHTLCCRR (SEQ ID NO:13), Alpha-defensin 5 (NP5): VFCTCRGFLCGSGERA SGSCTINGVRHTLCCRR (SEQ ID NO:14), and Alpha-defensin 6 (NP6): GICACR RRFCLNFEQFSGYCRVNGARYVR CCSRR (SEQ ID NO:15).

As used herein the term “rat alpha-defensin” refers to a polypeptide having one of the following amino acid sequences, allelic variants thereof, and pre-proproteins thereof. The sequence for rat alpha-defensins are as follows: Alpha-defensin 1 (RtNP1): VTCYCRRTRCGFRERLSGACGYRGRIYRLCCR (SEQ ID NO:16), Alpha-defensin 2 (RtNP2): VTCYCRSTRCGFRERLSGACGYRGRIYRLCCR (SEQ ID NO:17), Alpha-defensin 3 (RtNP3): CSCRTSSCRFGERLSGACRLNGRIYRLCC (SEQ ID NO:18), and Alpha-defensin 4 (RtNP4): ACYCRIGACVSGERLTGACGLNGRIYRLCCR (SEQ ID NO:19).

As used herein the term “mouse alpha-defensin” refers to a polypeptide having one of the following amino acid sequences, allelic variants thereof, and pre-proproteins thereof. The sequence for mouse alpha-defensins are as follows: Alpha-defensin 1 (MuCr1): LRDLVCYCRTRGCKRRERMNGTCRKGHLMYTLCCR (SEQ ID NO:20), Alpha-defensin 2 (MuCr2): LRDLVCYCRARGCKGRERMNGTCRKGHLL YMLCCR (SEQ ID NO:21), and Alpha-defensin 1α (MuCr1α): LRDLVCYCRKRGCK RRERMNGTCRKGHLMYTLCCR (SEQ ID NO:22).

As used herein, the term β-defensin refers to a polypeptide characterized as having anti-HIV activity as determined using an HIV virus inhibition assay, such as, the virus inhibition assay described herein. The beta-defensins are generally less than 100 amino acids long, usually 32-45 amino acids long, and characterized by a conserved cysteine motif and positive charge. (The conserved motif is apparent from SEQ ID NO:23 to SEQ ID NO:24 herein). Beta-defensins differ from alpha-defensins in their specific amino acid pattern, cysteine spacing, and disulfide connections (Kaiser et al., J. of Leukocyte Biology, 68:779-784 (2000)). Like alpha-defensins, the cysteines are dispersed throughout the oligopeptide, so that the cysteines provide the opportunity for extensive crosslinking, either intramolecularly or intermolecularly, covalently or noncovalently. The term “beta-defensin” embraces naturally occuring beta-defensins, i.e., polypeptides isolated from natural sources; synthetic beta-defensins, i.e., polypeptides chemically synthesized or produced by recombinant DNA techniques and having the amino acid sequence of a natural beta-defensin; and beta-defensin analogs, i.e., polypeptides having the biological activity of a beta-defensin and having an amino acid sequence that is not naturally occuring. The term also embraces pre-proprotein forms of beta-defensin that are processed intracellularly into a biologically active form.

Beta-defensins have been found in humans, cow, sheep, mice, pig, fowl, insects and plants and, in particular, in bovine tracheal mucosa, bovine neutrophils, bovine tongue, human neutrophils, vaginal and renal tissues, and epithelial cells, such as those of the skin, kidney, and trachea-bronchial lining (Diamond et al., Proc. Natl Acad. Sci. USA, 90:4596-4600, Selsted et al., J. Biol. Chem., 268:6641-6648, Singh et al., Proc. Natl. Acad. Sci. USA, 95:14961-14966, Bensch et al., FEBS Lett., 368:331-335, Schonwetter et al., Science, 3:267 1645-1648, and Lehrer et al., Annals New York Academy of Science, 228-239).

In certain embodiments, beta-defensins as defined above include polypeptides, pre-proproteins, processed proteins, polymorphic variants, alleles, interspecies homologs and mutants that have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 15, 20, 25, 30 or more amino acids, to an amino acid sequence encoded by a β-defensin nucleic acid as described herein, e.g., human β-defensin-1 or -2.

As used herein, the term “human beta-defensins” refers to a polypeptide having one of the following amino acid sequences, allelic variants thereof, and pre-proproteins thereof. The sequences for human beta-defensins 1 and 2 are as follows: Beta-defensin-1 (hBD-1): DHYNCVSSGGQCLYSACPIFTKIQGTCYR GKAKCC (SEQ ID NO:23), and Beta-defensin-1 (hBD-2): TCLKSGAICHPVFCPRRYK QIGTCGLPGTKCC (SEQ ID NO:24). Beta-defensin sequences are provided in PCT Publication No. WO 02/22686 and in Kaiser et al., Journal of Leukocyte Biology, 68:779-784 (2000).

The defensins of the present invention can be further characterized by their ability to (1) bind to antibodies, e.g., polyclonal antibodies, raised against an immunogen comprising an amino acid sequence of a defensin protein, and conservatively modified variants thereof; (2) specifically hybridize under stringent hybridization conditions to an anti-sense strand corresponding to a nucleic acid sequence encoding a defensin protein, and conservatively modified variants thereof; or (3) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a defensin nucleic acid. As previously mentioned, a defensin polynucleotide or polypeptide sequence is typically from a mammal including, but not limited to, primate, e.g., human; rodent, e.g., rat, mouse, hamster; cow, pig, horse, sheep, or any mammal. In an exemplary embodiment, the defensin is from humans. In a preferred embodiment, the defensin is human α-defensin-1, 2 or 3. The nucleic acids and proteins of the invention include both naturally occurring or recombinant molecules. A defensin protein of the present invention has anti-viral activity. Antiviral assays can be performed according to methods known to those of skill in the art and as described herein.

In vivo, defensins are made as precursors and processed from propeptides to yield mature peptides, typically with disulfide bridges. Defensin pre-propeptides are set forth in GenBank as Accession Numbers NP_{—}525128, AAH27917, CAC85520, CAC85511, AAG02237, CAC03097, AAF73853, NP_{—}066290, NP_{—}005208, NP_{—}001917, NP_{—}001916, NP_{—}004075, CAB65126, AAG22030, AAC69554, AAC51728, AAC50382, AAC33549, AAB59357, AAB49758, AAA52303, AAA35754, and AAM62424. The pre-propeptides are subsequently processed to mature defensins as described in Garcia et al., Cell Tissue Res., 306(2):257-264 (2001); Harder et al., J. Biol. Chem., 276(8):5707-5713 (2001); Jia et al., Gene, 263(1-2):211-218 (2001); Am. J. Pathol., 152(5):1247-1258 (1998); Daher et al., Proc. Natl. Acad. Sci. USA, 85(19):7327-7331 (1988); Mallow et al., J. Biol. Chem., 271(8):4038-4045 (1996); Wilde et al., J. Biol. Chem., 264(19):11200-11203 (1989); Gabay et al., Proc. Natl. Acad. Sci. USA, 86(14):5610-5614 (1989); Palfree et al., Mol. Endocrinol., 7(2):199-205 (1993); Selsted et al., J. Clin. Invest., 76(4):1436-1439 (1985); Mars et al., Blood, 71(6):1713-1719 (1988); Daher et al., Proc. Natl. Acad. Sci. USA, 85(19):7327-7331 (1988); Sparkes et al., Genomics, 5(2):240-244 (1989); Bateman et al., J. Biol. Chem., 266(12):7524-7530 (1991); Wagner et al., Genomics, 10(]): 114-125 (1991); Valore et al., Blood, 79(6):1538-1544 (1992); Lehrer et al., Annu. Rev. Immunol., 11:105-128 (1993); Linzmeier et al., FEBS Lett., 321(2-3):267-273 (1993); Harder et al., Am. J. Respir. Cell Mol. Biol., 22(6):714-721 (2000); Infect. Immun., 68(1):113-119 (2000); Genomics, 43(3):316-320 (1997); Mallow et al., J. Biol. Chem., 271(8):4038-4045 (1996); FEBS Lett., 315(2):187-192 (1993); McCray et al., Am. J. Respir. Cell. Molec. Biol., 16:343-349 (1997); Blood, 71(6):1713-1719 (1988); Jones et al., Biol. Chem., 267(32):23216-23225 (1992), Raj et al., FEMS Microbiology Letters, 206:9-18 (2002); Schutte et al., Proc. Natl. Acad. Sci. USA, 2129-2133 (2002); Hoover et al., J. Biol. Chem., 276(42):39021-39026; and Ganz et al., Eur. J. Haematol., 44(1):1-8.

Functionally, defensins are known to exhibit antibacterial and antifungal activity against a wide variety of microorganisms (Raj et al., FEMS Microbiology Letters, 206:9-18 (2002)). The positively charged defensins interact with negatively charged components of microbial membranes that include lipopolysaccharides in Gram-negative bacteria, polysaccharides in Gram-positive bacteria, and phospholipids. Defensins areknown to be secreted by phagocytic white cells involved in host defense against bacteria and fungi. Although the alpha-defensins are highly conserved, the minor variations are significant enough to alter the microbicidal potency and specificity of the various defensins (Mandal et al., J. Pept. Res., 59(3):95-104 (2002); Schibli et al., J. Biol. Chem, 277(10):8279-89 (2002); Garcia et al., Cell Tissue Res., 306(2):257-64; Fellermann et al., Eur. J. Gastroentero Hepatol., 13(7):771-776; Kagan et al., Toxicology, 87(1-3):131-149 (1994)).

Studies have also suggested that defensins play a role in fighting infection and stimulating immune responses. For example, defensins, and in particular beta-defensins 1 and 2, have been demonstrated to attract both immature dendritic cells and memory T cells involved in the initiation of primary and recall immune response (Ganz, Science, 286:420-421 (1999), Lillard et al., Proc. Natl. Acad. Sci. USA, 96:651-656 (1999)). With the present invention, it has now been discovered that defensins act as antiviral agents against HIV and can be used to treat and/or prevent HIV infection as well as other viral infections.

As explained above, naturally occurring, chemically synthesized, commercially available and recombinantly produced defensin polypeptides can be used in the compositions and methods of the present invention.

The known natural defensin polypeptides are described in the present application. It will be appreciated that additional natural defensin polypeptides useful in the methods of the present invention may be identified in the future from either the species set forth herein or other species.

### 2. Isolation from Naturally Occurring Sources

Naturally occurring defensins can be purified from any defensin source, e.g., from mycloid, airway, and intestinal cells, from human tissue such as neutrophils, pholmorphonuclear leukocytes, PBMC, or other phagocytic cells; and from epithelial tissue and other defensin-expressing tissue (see, e.g., van Wetering et al., Am. J. Physiol. 272:L888 (1997); Chertov et al., J. Biol. Chem. 271:2935-2940 (1996); and Raj et al., Biochem. J., 347:633-641 (2000)) as well as from defensin-expressing tissue of other mammals, such as rat and guinea pig. In an exemplary embodiment, naturally occurring defensins are purified from neutrophil and macrophage myeloid cells. Such purification methods include large scale purification, e.g., isolation of neutrophils from whole blood, e.g., obtained from a blood bank or medical waste products such as blast-relief apharesis from patients having acute myelogenous leukemia, and purification using known peptide purification methods for positively charged peptides. For example, the polypeptides may be purified using chromatographic procedures, such as reverse phase HPLC, gel permeation, ion exchange, e.g., anionic exchange, size exclusion, affinity, partition, or countercurrent distribution. Methods of purifying defensins are described in Raj et al., supra, and Ganz et al., J. Clin. Invest., 76:1427-1435 (1985).

Defensins can also be obtained using known cell culturing techniques to culture defensin-producing cells and to isolate the defensin molecules using the methods described herein. Suitable defensin-producing cell lines include, but are not limited to, CD8⁺, T lymphocytes, neutrophil and myeloid cell lines for human alpha-defensins 1-3, intestinal cell lines for human alpha-defensins 5-6, and endothelial cell lines for human beta-defensins 1-2. For general cell culturing techniques, see Freshney et al., Culture of Animal Cells (3rd ed. 1994).

Defensin molecules can also be obtained from commercial sources, such as Peptides International, American Peptides, and NJ Research, Inc.

This invention relies, in part, on routine techniques used in the field of recombinant genetics. Basic texts disclosing general recombinant methods suitable for use in this invention include, but are not limited to, Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

Defensin nucleic acids, polymorphic variants, orthologs, and alleles that are substantially identical to an amino acid sequence described herein can be isolated using defensin nucleic acid probes and oligonucleotides under stringent hybridization conditions, by screening libraries. Alternatively, expression libraries can be used to clone defensin protein, pre-proprotein, polymorphic variants, orthologs, and alleles by detecting expressed homologs immunologically with antisera or purified antibodies made against human defensin or portions thereof.

To make a cDNA library, one should choose a source that is rich in defensin RNA, e.g., PBMCs, polymorphonuclear leukocytes, neutrophils, lymphoblastoid cell lines, and other phagocytic cells. The cells can be primary cell or cell lines. The mRNA is then made into cDNA using reverse transcriptase, ligated into a recombinant vector, and transfected into a recombinant host for propagation, screening and cloning. Methods for making and screening cDNA libraries are well known (see, e.g., Gubler et al., Gene, 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then selected by gradient centrifugation and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating defensin nucleic acids and orthologs, alleles, mutants, polymorphic variants, and conservatively modified variants combines the use of synthetic oligonucleotide primers and amplification of an RNA or DNA template (see, U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of human defensins directly from mRNA, from cDNA, from genomic libraries or cDNA libraries. Degenerate oligonucleotides can be designed to amplify defensin homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of defensin encoding mRNA in physiological samples, for nucleic acid sequencing, or for other purposes. Genes amplified by the PCR reaction can be purified from agarose gels and cloned into an appropriate vector.

The gene for a defensin polypeptide may typically be cloned into intermediate vectors before transformation into prokaryotic or eukaryotic cells for replication and/or expression. These intermediate vectors are typically prokaryote vectors, e.g., plasmids, or shuttle vectors. Alternatively, homologous recombination can be used to activate a native defensin gene.

### 3. Chemical Synthesis and Purification of Defensin Polypeptides

Since the defensin polypeptides of the present invention are relatively short in length, they can be prepared using any of a number of chemical peptide synthesis techniques well known to those of ordinary skill in the art, including both solution methods and solid phase methods, with solid phase synthesis being presently preferred. A suitable approach for chemically synthesizing defensin polypeptides is disclosed by Raj et al, Biochem. J., 347:633-641 (2000), the teachings of which are incorporated herein by reference.

In particular, solid phase synthesis in which the C-terminal amino acid of the peptide sequence is attached to an insoluble support followed by sequential addition of the remaining amino acids in the sequence is the preferred method for preparing the defensin polypeptides of the present invention. Techniques for solid phase synthesis are described by Barany and Merrifield, Solid-Phase Peptide Synthesis, in The Peptides: Analysis, Synthesis, Biology (Gross and Meienhofer (eds.), Academic Press, N.Y., vol. 2, pp. 3-284 (1980)); Merrifield et al., J. Am. Chem. Soc., 85:2149-2156 (1963); and Stewart et al., Solid Phase Peptide Synthesis (2nd ed., Pierce Chem. Co., Rockford, Ill. (1984)).

Solid phase synthesis is started from the carboxy-terminal end (i.e., the C-terminus) of the peptide by coupling a protected amino acid via its carboxyl group to a suitable solid support. The solid support used is not a critical feature of the present invention provided that it is capable of binding to the carboxyl group while remaining substantially inert to the reagents utilized in the peptide synthesis procedure. For example, a starting material can be prepared by attaching an amino-protected amino acid via a benzyl ester linkage to a chloromethylated resin or a hydroxymethyl resin or via an amide bond to a benzhydrylamine (BHA) resin or p-methylbenzhydrylamine (MBHA) resin. Materials suitable for use as solid supports are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

The acid form of the peptides of the present invention may be prepared by the solid phase peptide synthesis procedure using a benzyl ester resin as a solid support. The corresponding amides may be produced by using benzhydrylamine or methylbenz-hydrylamine resin as the solid support. Those skilled in the art will recognize that when the BHA or MBHA resin is used, treatment with anhydrous hydrofluoric acid to cleave the polypeptide from the solid support produces a polypeptide having a terminal amide group.

The α-amino group of each amino acid used in the synthesis should be protected during the coupling reaction to prevent side reactions involving the reactive α-amino function. Certain amino acids also contain reactive side-chain functional groups (e.g., sulfhydryl, amino, carboxyl, hydroxyl, etc.) which must also be protected with appropriate protecting groups to prevent chemical reactions from occurring at those sites during the polypeptide synthesis. Protecting groups are well known to those of skill in the art. See, for example, The Peptides: Analysis, Synthesis, Biology, Vol. 3: Protection of Functional Groups in Peptide Synthesis (Gross and Meienhofer (eds.), Academic Press, N.Y. (1981)).

The peptide can be cleaved and the protecting groups removed by stirring the insoluble carrier or solid support in anhydrous, liquid hydrogen fluoride (HF) in the presence of anisole and dimethylsulfide at about 0° C. for about 20 to 90 minutes, preferably 60 minutes; by bubbling hydrogen bromide (HBr) continuously through a 1 mg/10 mL suspension of the resin in trifluoroacetic acid (TFA) for 60 to 360 minutes at about room temperature, depending on the protecting groups selected; or, by incubating the solid support inside the reaction column used for the solid phase synthesis with 90% trifluoroacetic acid, 5% water and 5% triethylsilane for about 30 to 60 minutes. Other deprotection methods well known to those of skill in the art may also be used.

There are six cysteines residues present in the known naturally occurring alpha-defensins. The disulfide linkages formed by the cysteine residues are between the first and sixth cysteine, the second and fourth cysteine, and the third and fifth cysteine, wherein the cysteines are numbered one through six from the N-terminus to the C-terminus of the peptide. Additionally, there are six cysteine residues present in most of the known beta-defensins. The disulfide linkages of the beta-defensins are between the first and fifth cysteine, the second and fourth cysteine, and the third and sixth cysteine, wherein the cysteines are numbered one through six from the N-terminus to the C-terminus of the peptide

The formation of the disulfide bonds between the cysteine residues present in the defensin molecules can be carried out using known methods. For example, in Raj et al., supra, three distinct thiol protecting groups and a three stage process is used to form the disulfide bonds in order to minimize undesired side-products. In order to provide the proper orientation for bond formation, a disulfide bridge is first formed between a N and C terminal cysteine (See Hill et al., Science, 25:1481-1485 and Pardi et al., Biochemistry, 31:11357-11364). In other methods, simultaneous oxidation of the six thiol groups, or a two stage process for disulfide bond formation is performed (See Khan, Methods Enzymol., 61:339-378 and Lauth et al., Insect Biochem. Mol. Biol., 28:1059-1066).

The cationic polypeptides, i.e., defensin polypeptides, of the present invention can be isolated and purified from the reaction mixture by means of peptide purification methods well known to those of skill in the art, e.g., purification methods described above.

It will be readily apparent to those of skill in the art that a number of modifications (e.g., additions, deletions or substitutions) can be made to the defensin polypeptides used in the compositions and methods of the present invention. For instance, it has been found that additional amino acids can be added to the N-terminal and/or C-terminal of the defensin polypeptides to increase their activity (see, Raj et al, Biochem. J., 347:633-641 (2000)). Moreover, the defensin polypeptides can be modified to remove protease cleavage sites (e.g., trypsin and chymotrypsin cleavage sites) (see, e.g., Selsted et al., J. Clin. Invest., 76:1436-1439 91985). Typically, this is done by replacing the amino acid recognized by the protease (e.g., arginine) with an amino acid of like-kind that is not recognized by the protease. Such modifications (e.g., additions, deletions and substitutions) fall within the scope of the present invention in that such modified defensin polypeptides can be used in the compositions and methods of the present invention.

It will also be readily apparent to those of skill in the art that known modification procedures can be used to generate active defensin polypeptides with one or more amino acid difference from the known naturally occurring defensin polypeptides. The defensins can be assayed for defensin activity using one of the assays described herein. Those defensin polypeptides having anti-HIV activity can be used in the methods and compositions of the present invention. Exemplary modification methods are site-directed mutagenesis, point mismatch repair, or oligonucleotide directed mutagenesis.

Site directed mutagenesis is well known in the art and is described in the following references, e.g., Ling et al., Anal. Biochem., 254(2):157-178 (1997); Dale et al., Methods Mol. Biol., 57:369-374 (1996); Smith, Ann. Rev. Genet., 19:423-462 (1985); Botstein et al., Science, 229:1193-1201 (1985); Carter, Biochem. J., 237:1-7 (1986); and Kunkel, Nucleic Acids & Molecular Biology (Eckstein, F. and Lilley, D. M. J. eds., Springer Verlag, Berlin (1987)); mutagenesis using uracil containing templates (Kunkel, Proc. Natl. Acad. Sci. USA, 82:488-492 (1985); Kunkel et al., Methods in Enzymol., 154:367-382 (1987); and Bass et al., Science, 242:240-245 (1988)); oligonucleotide-directed mutagenesis (Methods in Enzymol., 100:468-500 (1983); Methods in Enzymol., 154:329-350 (1987); Zoller et al., Nucleic Acids Res., 10:6487-6500; Zoller et al., Methods in Enzymol., 100:468-500; and Zoller et al., Methods in Enzymol., 154:329-350); phosphorothioate-modified DNA mutagenesis (Taylor et al., Nuc. Acids Res., 13:8749-8764 (1985); Taylor et al., Nucl. Acids Res., 13:8765-8787 (1985); Nakamaye et al., Nucl. Acids Res., 14: 9679-9698 (1986); Sayers et al., Nucl. Acids Res., 16:791-802 (1988); and Sayers et al., Nucl. Acids Res., 16:803-814 (1988)); mutagenesis using gapped duplex DNA (Kramer et al., Nucl. Acids Res., 12:9441-9456 (1984); Kramer et al., Methods in Enzymol., 154:350-367 (1987); Kramer et al., Nucl. Acids Res., 16:7207 (1988); and Fritz et al., Nucl. Acids Res., 16:6987-6999 (1988)).

An additional modification method well known in the art is point mismatch repair, e.g., (Kramer et al., Cell, 38:879-887 (1984)), mutagenesis using repair-deficient host strains (Carter et al., Nucl. Acids Res., 13:4431-4443 (1985); and Carter, Methods in Enzymol., 154:382-403 (1987)), deletion mutagenesis (Eghtedarzadeh et al., Nucl. Acids Res., 14:5115 (1986)), restriction-selection and restriction-selection and restriction-purification (Wells et al., Phil. Trans. R. Soc. Lond., A317: 415-423 (1986)), mutagenesis by total gene synthesis (Nambiar et al., Science, 223:1299-1301 (1984); Sakamar et al., Nucl. Acids Res., 14:6361-6372 (1988); Wells et al., Gene, 34:315-323 (1985); and Grundström et al., Nucl. Acids Res., 13:3305-3316 (1985)), double-strand break repair (Mandecki (1986); Arnold, Current Opinion in Biotechnology, 4:450-455 (1993). “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology, Volume 154, which also describes useful controls for trouble-shooting problems with various mutagenesis methods.

Oligonucleotide directed mutagenesis can also be used to introduce site-specific mutations in a nucleic acid sequence of interest. Examples of such techniques are described in the references set forth above and in, e.g., in Reidhaar-Olson et al., Science, 241:53-57 (1988). Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

### 4. Purification of Recombinant Defensins

Recombinant defensins can be purified from any suitable expression system, as described below. The defensin protein can be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when a recombinant defensin protein is being purified. For example, proteins having established molecular adhesion properties can be reversibly fused to the defensin protein. With the appropriate ligand or substrate, e.g., anti-defensin antibodies or a negatively charged substate, defensin proteins can be selectively adsorbed to a purification column and then freed from the column in a relatively pure form. The fused protein is then removed by enzymatic activity. Finally, defensin proteins can be purified using immunoaffinity columns. Recombinant defensin proteins can be purified from any suitable source, include yeast, insect, bacterial, and mammalian cells.

Recombinant proteins may be expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria' cells are used for isolation of protein.

Proteins expressed in bacteria may form insoluble aggregates (“inclusion bodies”). Several protocols are suitable for purification of defensin protein inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. Human defensin proteins are separated from other bacterial proteins by standard separation techniques, e.g., with Ni-NTA agarose resin.

Alternatively, it is possible to purify defensin proteins from bacteria periplasm. After lysis of the bacteria, when the defensin protein is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### 5. Standard Protein Separation Techniques for Purifying Recombinant Defensin Proteins

### a) Solubility Fractionation

Often as an initial step, particularly if the defensin protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

### b) Size Differential Filtration

The molecular weight of the defensin proteins can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant defensin protein will pass through the membrane into the filtrate. The filtrate can then be chromatographed as described below.

### c) Column Chromatography

The defensin proteins can also be separated from other proteins on the basis of its size, net surface charge, hydrophobicity, and affinity for ligands. In addition, antibodies raised against defensin proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### D. Methods of Inhibiting HIV and Viral Infection and Replication—“Therapies for HIV Infection and AIDS”

As described herein, defensin proteins, nucleic acids encoding defensin proteins, and small organic molecules, or fragments thereof,that increase defensin activity or expression can be used to inhibit HIV, e.g., by killing HIV infected cells or by inhibiting HIV replication. Defensins can be used therapeutically or prophylactically in a person. For instance, defensins can be used to inhibit HIV production by infected CD4⁺ cells. The defensin proteins, nucleic acids encoding defensin proteins, and small organic molecules can also be used to stop progression to AIDS in an HIV infected subject. The defensin proteins, nucleic acids encoding defensin proteins, and small organic molecules can also be used prophylactically, e.g., after exposure or suspected exposure to HIV to prevent infection or to kill infected cells, or to prevent transmission, e.g., maternal transmission of the virus to the fetus.

Methods known in the art for therapeutic delivery of nucleic acids and proteins to an individual can be used in the methods of the present invention for treating or preventing HIV infection in a subject, e.g., cellular transfection, gene therapy, direct administration with a delivery vehicle or pharmaceutically acceptable carrier, indirect delivery by providing recombinant cells or regulatory sequences that enhance production of endogenous protein. For example, as explained above, the present invention demonstrates for the first time that CD8⁺ cells produce alpha-defensins which have been identified as being responsible for the CAF activity previously seen. As such, defensins can be delivered to a person by administering to the person CD8⁺ cells that have been stimulated to produce defensins. In one method, CD8⁺ cells are expanded ex vivo by any of the means known to expand such cells. The cell cultures are then tested to determine if the cells are producing defensins. Thereafter, cells that are producing an appropriate amount of defensin are introduced into a person. The cells can be derived from the individual to be treated or, alternatively, they can be from another individual. However, it is preferred to administer cells that are HLA-matched to avoid provoking an immune response against the cells.

Standard assays for measuring HIV infection and progression to AIDS can be used to determine whether a subject is positively responding to defensin therapy. For example, after defensin administration, a subject's T cell count can be monitored. A rise in T cells indicates that the subject is benefiting from defensin administration. Additionally, the “endogenous assay” or “acute infection assay” as described in Levy et al., Immunology Today 17, 5:223 (1996), can be used to measure the anti-HIV response of CD8⁺ cells in a subject. For example, in the acute infection assay, CD4⁺ cells from uninfected individuals are acutely infected with HIV and are cultured with CD8⁺ cells from infected individuals at different CD8⁺/CD4⁺ cell ratios. The antiviral effect is determined by the extent of reduction in virus production. The extent of reduction in virus production by recombinant CD8⁺ cells of the present invention can be used to determine the extent to which the methods of the present invention are effective at inhibiting virus production in a subject

As explained above, it has now been discovered that defensin polypeptides have anti-viral activity. As such, defensin polypeptides can be used to inhibit a wide variety of viruses and, thus, to treat a wide variety of viral infections in a human. Viruses that can be inhibited using defensin polypeptides include, but are not limited, to DNA viruses, RNA viruses as well as retroviruses. Examples of viruses that can be inhibited using defensin polypeptides include, but are not limited to, Herpes viruses, Hepatitis (A, B and C) viruses, influenza viruses, POX viruses, Rhino viruses and HTLV (Human T-cell Leukemia) viruses (e.g., HTLV 1 and 2). Based on their anti-viral activity, those of skill in the art will be aware of other viruses that can be inhibited using defensin polypeptides.

### 1. Cellular Transfection and Gene Therapy

The present invention provides the nucleic acid sequences of the defensin proteins which can be used for the transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses a defensin protein of the present invention, thereby mitigating the effects of low, absent, partial inactivation, or abnormal expression of a defensin gene, particularly as it relates to viral infection, e.g., HIV. The compositions are administered (e.g., by injection into a muscle) to a patient in an amount sufficient to elicit a therapeutic response in the patient. An amount adequate to accomplish this is defined as “therapeutically effective dose or amount.”

In another aspect, the present invention provides a method of inhibiting HIV infection in a human comprising transfecting a cell with a nucleic acid comprising a nucleotide sequence encoding a defensin polypeptide wherein the nucleic acid comprises an inducible promoter operably linked to the nucleotide sequence encoding the defensin. In one embodiments, expression of defensin proteins from eukaryotic vectors can be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal. Inducible expression vectors are often chosen if expression of the protein of interest is detrimental to eukaryotic cells. Other regulatory structures, e.g., enhancers, can be used to increase expression of the defensin polypeptide.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies (for a review of gene therapy procedures, see Anderson, Science, 256:808-813 (1992); Nabel et al., TIBTECH, 11:211-217 (1993); Mitani et al., TIBTECH, 11:162-166 (1993); Mulligan, Science, 926-932 (1993); Dillon, TIBTECH, 11:167-175 (1993); Miller, Nature, 357:455-460 (1992); Van Brunt, Biotechnology, 6(10):1149-1154 (1998); Vigne, Restorative Neurology and Neuroscience, 8:35-36 (1995); Kremer et al., British Medical Bulletin, 51(1):31-44 (1995); Haddada et al., in Current Topics in Microbiology and Immunology (Doerfler & Böhm eds., 1995); and Yu et al., Gene Therapy, 1:13-26 (1994)).

In another aspect of the present invention, defensin nucleic acids are not introduced into a cell by transfection, but instead an exogenous regulator sequence, e.g., promoter or enhancer, an exogenous exon, either coding or noncoding, and a splice donor site are introduced into a preselected site in the genome for homologous recombination with a defensin gene. Using these methods, exogenously supplied expression sequences recombine with genomic DNA allowing defensins to be produced in human cells using the naturally-occurring endogenous exons encoding these proteins. Such methods are described in U.S. Pat. No. 5,733,746, which issued to Treco et al.

### 2. Pharmaceutical Compositions and Administration

Pharmaceutically acceptable carriers are determined in part by the particular composition being administered (e.g., nucleic acid, protein, modulatory compounds such as small organic molecules or transduced cell), as well as by the particular method used to administer the composition. The present invention encompasses delivery of pharmaceutical compositions comprising proteins, nucleic acids, and small organic molecules, and the like. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, transdermal application, or rectal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit- or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

An important factor in the administration of polypeptide compounds is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins and other compounds such as liposomes have been described, which have the ability to translocate polypeptides across a cell membrane.

In addition, it can be important to alter the properties of the polypeptide, so that it is less “sticky” and does not form aggregates or bind to other proteins, or so that it has enhanced pharmacokinetics (e.g., a longer half life) and is more stable, e.g., in serum or while being stored, e.g., by PEGylating the protein or conjugating the protein with a lipid or other moiety. It may also be important to provide a signal peptide, leader sequence, or a secretory peptide to the protein, for secretion in vivo or in vitro. Defensin polypeptides of the invention can also be specifically targeted to cells using a targeting moiety, such as a ligand that binds to a cell surface molecule of a CD4⁺ cell, e.g., antibodies, ligands, receptors, etc. Accordingly, the present invention provides delivery vehicles for the defensins described herein, as well as fusion proteins that have a heterologous moiety that has the ability to stabilize, specifically deliver or target, secrete, provide a detectable label, etc. for the defensin protein. In one embodiment, the fusion protein comprises an albumin moiety and a defensin moiety. The albumin moiety can be at least a portion of albumin (e.g., human albumin) sufficient to extend the life of the defensin. Such constructs are described in more detail in International PCT Publication No. WO 01/79480, Oct. 25, 2001 (Rosen et al., “Albumin Fusion Proteins”).

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology, 6:629-634 (1996)). Another subsequence, the h (hydrophobic) domain of signal peptides, was found to have similar cell membrane translocation characteristics (see, e.g., Lin et al., J. Biol. Chem., 270(1):4255-14258 (1995)).

Examples of peptide sequences include, but are not limited to an 11 amino acid peptide of the tat protein of HIV; a 20 residue peptide sequence which corresponds to amino acids 84-103 of the p16 protein (see Fahraeus et al., Current Biology, 6:84 (1996)); the third helix of the 60-amino acid long homeodomain of Antennapedia (Derossi et al., J. Biol. Chem., 269:10444 (1994)); the h region of a signal peptide such as the Kaposi fibroblast growth factor (K-FGF) h region (Lin et al., supra); or the VP22 translocation domain from HSV (Elliot et al., Cell, 88:223-233 (1997)). Other suitable chemical moieties that provide enhanced cellular uptake can also be chemically linked to the defensins of the invention.

Toxin molecules also have the ability to transport polypeptides across cell membranes. Often, such molecules (called “binary toxins”) are composed of at least two parts: (1) a translocation or binding domain or polypeptide, and (2) a separate toxin domain or polypeptide. Typically, the translocation domain or polypeptide binds to a cellular receptor, and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used in attempts to deliver peptides to the cell cytosol as internal or amino-terminal fusions (Arora et al., J. Biol. Chem., 268:3334-3341 (1993); Perelle et al., Infect. Immun., 61:5147-5156 (1993); Stenmark et al., J. Cell Biol., 113:1025-1032 (1991); Donnelly et al., PNAS, 90:3530-3534 (1993); Carbonetti et al., Abstr. Annu. Meet. Am. Soc. Microbiol., 95:295 (1995); Sebo et al., Infect. Immun., 63:3851-3857 (1995); Klimpel et al., PNAS U.S.A., 89:10277-10281 (1992); Novak et al., J. Biol. Chem., 267:17186-17193 1992), and U.S. Pat. Nos. 5,602,095, 4,892,827, and 5,608,039).

Such subsequences can be used to translocate defensins across a cell membrane. Defensins can be conveniently fused to or derivatized with such sequences. Typically, the translocation sequence is provided as part of a fusion protein. Optionally, a linker can be used to link the defensin and the translocation sequence. Any suitable linker can be used, e.g., a peptide linker or other chemical linkers.

The defensin can also be introduced into an animal cell, preferably a mammalian cell, via a microparticles and liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell.

The liposome fuses with the plasma membrane, thereby releasing the drug into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle. Liposome membranes can be constructed so that they become destabilized when the environment becomes acidic near the liposome membrane (see, e.g., PNAS, 84:7851 (1987); Biochemistry, 28:908 (1989)). When liposomes are endocytosed by a target cell, for example, they become destabilized and release their contents. This destabilization is termed fusogenesis. Dioleoylphosphatidylethanolamine (DOPE) is the basis of many “fusogenic” systems.

Such liposomes typically comprise a defensin of choice and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes as described in, e.g., Szoka et al., Ann. Rev. Biophys. Bioeng., 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91/17424, Deamer et al., Biochem. Biophys. Acta, 443:629-634 (1976); Fraley, et al., PNAS, 76:3348-3352 (1979); Hope et al., Biochem. Biophys. Acta, 812:55-65 (1985); Mayer et al., Biochem. Biophys. Acta, 858:161-168 (1986); Williams et al., PNAS, 85:242-246 (1988); Liposomes (Ostro (ed.), 1983, Chapter 1); Hope et al., Chem. Phys. Lip., 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments of the present invention, it is desirable to target the liposomes of the invention using targeting moieties that are specific to a particular cell type, tissue, and the like. Targeting of liposomes using a variety of targeting moieties (e.g., ligands, receptors, and monoclonal antibodies) has been previously described (see, e.g., U.S. Pat. Nos. 4,957,773 and 4,603,044).

Standard methods for coupling targeting agents to liposomes can be used. These methods generally involve incorporation into liposomes lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A (see, Renneisen et al., J. Biol. Chem., 265:16337-16342 (1990) and Leonetti et al., PNAS, 87:2448-2451 (1990).

In another embodiment, the defensin polypeptides of the present invention can be used as an adjuvant. More particularly, defensin polypeptides can be used as adjuvants to boost immunogenecity. As such, defensin polypeptides or nucleic acids encoding such defensin polypeptides can be used in combination with vaccines or other antigens to increase the antigenic response.

The methods of the present invention treat or prevent HIV infection and AIDS in a subject. The amount of defensin adequate to accomplish this is defined as a “therapeutically effective dose”. Single or multiple administrations of defensins or defensin formulations can be administered depending on the dosage and frequency as required and tolerated by the patient. The formulations should provide a sufficient quantity of active agent, i.e., defensin, to effectively treat or prevent HIV infection and AIDS in a subject.

The dose administered to a patient, in the context of the present invention should be sufficient to effect a beneficial therapeutic response in the patient over time. The dose will be determined by the efficacy of the particular delivery method employed, the composition to be administered, such as nucleic acid, polypeptide, or small organic molecule, and the condition of the patient, as well as the body weight or surface area of the patient to be treated. The size of the dose also will be determined by the existence, nature, and extent of any adverse side-effects that accompany the administration of a particular defensin composition, or transduced cell type in a particular patient.

For administration, compounds of the present invention can be administered at a rate determined by the LD-50 of the candidate compound, and the side-effects of the candidate compound at various concentrations, as applied to the mass and overall health of the patient. Administration can be accomplished via single, multiple, or divided doses. In general, the dosages will range from 1 microgram to 100 mg per kg of body weight, from 1 microgram to 1 mg per kg of body weight, or from 10 micrograms to 10 mg per kg of body weight. Those of skill would understand that other ranges may be suitable and could readily be ascertained. For example, a particular composition may be more effective at higher or lower doses. By evaluating a patient using the methods described herein, a skilled practitioner will be able to determine whether a patient is responding to treatment and will know how to adjust the dosage levels accordingly.

### 3. Combination Therapies

In numerous embodiments, the defensin polypeptides of the present invention may be administered in combination with one or more additional compounds or therapies. For example, multiple defensin polypeptides can be co-administered, or one or more defensin polypeptides can be administered in conjunction with one or more therapeutic compounds. In one embodiment, the other therapeutic agent is one that is used to prevent or treat HIV infection. In another embodiment, the other therapeutic agent is an agent used to treat an opportunistic infection associated with HIV infection and/or to treat or prevent HIV infection.

Suitable therapeutic agents for use in combination with the defensin polypeptides of the present invention include, but are not limited to, protease inhibitors, nonnucleoside reverse transcriptase inhibitors, nucleoside reverse transcriptase inhibitors, antiretroviral nucleosides, fusion inhibitors, entry inhibitors as well as other anti-viral agents effective to inhibit or treat HIV infection. Further examples of suitable therapeutic agents include, but are not limited to, zidovudine, didanosine, stavudine, interferon, lamivudine, adefovir, nevirapine, delaviridine, loviride, saquinavir, indinavir, AZT, T20, T22 and T2. Other suitable therapeutic agents include, but are not limited to, antibiotics or other anti-viral agents, e.g., acyclovir. In another embodiment, the defensin polypeptide or polypeptides are co-administered with a soluble form of HIV co-receptor, such as CXCR4 of CCR5. Naturally, the co-receptors are membrane proteins comprising an extra-cellular portion, a trans-membrane portion and an intra-cellular portion. They can be made soluble by eliminating the transmembrane and intracellular portion. These portions can be eliminated recombinantly and the extra-cellular portion can thus be expressed as a recombinant protein either alone or as part of a fusion protein.

Other combination therapies known to those of skill in the art can be used in conjunction with the methods of the present invention.

### E. Diagnostic and Prognostic Applications

Defensin molecules of the invention, e.g., nucleic acids and proteins, are also useful for diagnostics and prognostics. For example, increased defensin levels in biological samples (e.g., tissues or serum samples) are predictive of delayed or non-progression to AIDS or T cell counts below a level such as 200. In one embodiment, CD8⁺ cells are isolated, stimulated in vitro, and defensin levels are measured. Increased defensin levels, e.g., in human tissue such as neutrophils, are correlated with long term survival of HIV infected subjects. Accordingly, by detecting defensin levels in an individual, a determination can be made on the disease state of an individual. As explained herein, it has now been discovered that an individual having increased defensin levels is less likely to progress to AIDS as compared to an individual with lower defensin levels. Defensin levels can also be used to determine choice of anti-viral medicine, dosages, and duration of treatment. Therefore,,methods known to those of skill in the art for detection of nucleic acids and proteins can be used for diagnosis and prognosis of HIV infection and progression to AIDS, e.g., PCR, northern and Southern blots, reverse transcription and amplification of mRNA, isolation of total RNA or poly A⁺ RNA, dot blots, nucleic acid arrays, western blots, in-situ hybridization, immunoassays such as immunoprecipitation, ELISA, proteomics assays, polynucleotide array technology and the like.

For example, methods for using arrays of nucleic acid probes for monitoring expression of mRNA populations are described in U.S. Pat. No. 6,040,138, EP Patent No. 853,679 and PCT Publication No. WO97/27317. Such methods employ groups of probes complementary to mRNA target sequences of interest. An mRNA populations or an amplification product thereof is applied to such an array, and targets of interest are identified, and optionally, quantified from the extent of specific binding to complementary probes. Optionally, binding of target to probes known to be mismatched with the target can be used as a measure of background nonspecific binding and subtracted from specific binding of target to complementary probes.

Accordingly, nucleic acids encoding defensin proteins can be used with oligonucleotide array technology, high density or low density (e.g., GeneChip™) to identify cDNA encoding defensin proteins, orthologs, alleles, conservatively modified variants, and polymorphic variants in this invention. In the case where the homologs being identified are linked to modulation of viral infection, they can be used with GeneChip™ arrays as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

### 1. Immunological Detection of Defensin Polypeptides

In addition to the detection of defensin gene and gene expression using nucleic acid hybridization technology, immunoassays can also be used to detect defensin proteins of the invention. Such assays are useful for screening for modulators of defensin, as well as for therapeutic and diagnostic applications. Immunoassays can be used to qualitatively or quantitatively analyze defensin protein. A general overview of the applicable technology can be found in Harlow & Lane, Antibodies: A Laboratory Manual (1988).

Methods of producing polyclonal and monoclonal antibodies that react specifically with the defensin proteins are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science, 246:1275-1281 (1989); Ward et al., Nature, 341:544-546 (1989)).

A number of immunogens comprising portions of defensin protein may be used to produce antibodies specifically reactive with defensin protein. For example, recombinant or chemically synthesized defensin protein or an antigenic fragment thereof, can be isolated as described herein. Recombinant protein can be expressed in eukaryotic or prokaryotic cells as described above, and purified as generally described above. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler et al., Eur. J. Immunol., 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate. DNA sequences which encode a monoclonal antibody or a binding fragment thereof by screening a DNA library from human B cells according to the general protocol outlined by Huse, et al., Science, 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non- defensin proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular defensin ortholog, such as human defensin, can also be made, by subtracting out other cross-reacting orthologs from a species such as a non-human mammal. In this manner, antibodies that bind only to defensin protein may be obtained.

Once the specific antibodies against defensin protein are available, the protein can be detected by a variety of immunoassay methods. In addition, the antibody can be used therapeutically as a defensin modulators. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case the defensin protein or antigenic subsequence thereof). The antibody (e.g., anti-defensin) may be produced by any of a number of means well known to those of skill in the art and as described above.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize defensin protein, or secondary antibodies that recognize anti-defensin.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by the use of electronic detectors such as charge coupled devices (CCDs) or photomultipliers and the like. Similarly, enzymatic labels may be detected by providing the appropriate substrates for the enzyme and detecting the resulting reaction product. Colorimetric or chemiluminescent labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### 2. SELDI Detection of Defensin Polypeptides

In addition to the detection of defensin gene and gene expression using nucleic acid hybridization technology and immunologic techniques, Surface-enhanced laser desorption/ionization (“SELDI”) can be used to detect the defensins of the present invention or compounds that modulate or bind to defensins. SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. In some embodiments, SELDI involves the capture of analyte molecules on a solid support, such as a mass spectrometry probe surface, derivatized with an adsorbent that captures target analytes from a mixture. Typically a matrix material is applied to the captured analytes and the analytes are detected by laser desorption mass spectrometry. The adsorbents used can be chromatographic or biospecific. SELDI technology is described in, e.g., U.S. Pat. No. 5,719,060 (Hutchens and Yip) and U.S. Pat. No. 6,225,047 (Hutchens and Yip).) SELDI-based biochips and instruments are available from Ciphergen Biosystems, Inc., Fremont, Calif.

“Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidazole are useful reactive moieties to covalently bind biospecific adsorbents such as antibodies or cellular receptors.

“Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-methoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 5,719,060 and U.S. patent application Ser. No. 60/408,255, filed Sep. 4, 2002 (Kitagawa, “Monomers And Polymers Having Energy Absorbing Moieties Of Use In Desorption/Ionization Of Analytes”).

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Adsorption” refers to detectable non-covalent binding of an analyte to an adsorbent or capture reagent. “Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature. “Solid support” refers to a solid material which can be derivatized with, or otherwise attached to, a chemical moiety, such as a capture reagent, a reactive moiety or an energy absorbing species. Exemplary solid supports include chips (e.g., probes), microtiter plates and chromatographic resins. “Chip” refers to a solid support having a generally planar surface to which a chemical moiety may be attached. Chips that are adapted to engage a probe interface are also called “probes.” “Biochip” refers to a chip to which a chemical moiety is attached. Frequently, the surface of the biochip comprises a plurality of addressable locations, each of which location has the chemical moiety attached there. “Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International PCT Publication No. WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International PCT Publication No. WO 00/56934 (Englert et al., “Continuous porous matrix arrays,” Sep. 28, 2000).

In one embodiment of the present invention, defensins can be detected on both chromatographic and biospecific SELDI biochips. It has been found that the defensins are resolved well on SELDI biochips having cation exchange surfaces (WCX2 ProteinChip array, Ciphergen Biosystems, Inc.). The sample is applied to the chip and allowed to incubate to promote adsorption. Then the biochip is washed with a 100 mM sodium acetate, pH 4.5, with 0.2% Triton-X 100 in PBS buffer. Proteins are detected by mass spectrometry. The alpha-defensins appear as a triplet of peaks at about 3371 D, 3443 D and 3484 D.

Alternatively, the defensins can be detected on SELDI biochips having defensin-specific antibodies attached to their surface. In one embodiment, a SELDI biochip comprising functional groups such as carboxodiimizole or epoxide (e.g., a PS10 or a PS20 ProteinChip array, Ciphergen Biosystems, Inc.) is derivatized with the antibodies by incubating the antibodies on the biochip surface in a reaction buffer. Then the sample is applied to the chip surface, incubated to promote binding, and washed. Proteins are detected by mass spectrometry.

Upon capture on a biochip, the defensins (or modulators of defensins as well as compounds that bind defensins) can be detected using mass spectrometry and, in particular, SELDI as described above. Data generation in mass spectrometry begins with the detection of ions by an ion detector. A typical laser desorption mass spectrometer can employ a nitrogen laser at 337.1 nm. A useful pulse width is about 4 nanoseconds. Generally, power output of about 1-25 μJ is used. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

A computer can transform the resulting spectrum into various formats for displaying. In one format, referred to as “spectrum view or retentate map,” a standard spectral view can be displayed, wherein the view depicts the quantity of analyte reaching the detector at each particular molecular weight. In another format, referred to as “peak map,” only the peak height and mass information are retained from the spectrum view, yielding a cleaner image and enabling analytes with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique analytes and analytes that are up- or down-regulated between samples.

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

### F. Assays for Measuring HIV-Inhibition

The level of virus in a cell culture, cell or whole organism is measured by means known in the art. Typically, the level of virus is measured in a western blot or other immunoassay such as an ELISA, or by performing quantitative PCR. In immunoassay formats, the level of virus is measured by monitoring the amount of a viral protein (or viral capsid) by quantifying binding of the protein to an immunogenic reagent such as an antibody. In quantitative PCR, the level of a viral nucleic acid is measured by monitoring PCR amplification products, and comparing the amount of amplified nucleic acid obtained, as compared to a amplification products obtained from amplification performed on a known reference nucleic acid.

Methods of producing polyclonal and monoclonal antibodies are known to those of skill in the art, and many anti-viral antibodies are commercially available. See, e.g., Coligan (1991) Current Protocols in Immunology Wiley/Greene, NY; and Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497. Such techniques include antibody preparation by selection of antibodies from libraries of recombinant antibodies in phage or similar vectors. See, Huse et al. (1989) Science 246: 1275-1281; and Ward, et al. (1989) Nature 341: 544-546. Specific monoclonal and polyclonal antibodies and antisera will usually bind with a K_{D} of less than about 0.1 mM, more usually less than about 1 μM, preferably less than about 0.1 μM or better, and most typically and preferably, 0.01 μM or better.

Frequently, the polypeptides and their corresponding antibodies will be labeled by joining, either covalently or non covalently, a substance which provides for a detectable signal. A wide variety of labels and conjugation techniques are known and are reported extensively in both the scientific and patent literature. Suitable labels include radionucleotides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241.

In one preferred class of embodiments, the viral proteins detected when quantifying viral inhibition in the present invention are used for the detection of the virus (such as HIV) in human patients. For instance, HIV polypeptides are used routinely in western blots for the detection of antibodies to HIV in a patient's blood, and the reciprocal experiment (for detecting HIV polypeptides in a patient's blood) is suitable for measuring HIV viral load in a patient's blood. Such tests are well known, and are presently a standard method by which HIV-1 and HIV-2 infections are detected in patient populations. A variety of immunoassay formats are known and available.

A particular protein can be quantified by a variety of immunoassay methods. For a review of immunological and immunoassay procedures in general, see Stites and Terr (eds.) 1991 Basic and Clinical Immunology (7th ed.). Moreover, the immunoassays of the present invention can be performed in any of several configurations, e.g., those reviewed in Maggio (ed.) (1980) Enzyme Immunoassay CRC Press, Boca Raton, Fla.; Tijan (1985) “Practice and Theory of Enzyme Immunoassays,” Laboratory Techniques in Biochemistry and Molecular Biology, Elsevier Science Publishers B. V., Amsterdam; Harlow and Lane, supra; Chan (ed.) (1987) Immunoassay: A Practical Guide Academic Press, Orlando, Fla.; Price and Newman (eds.) (1991) Principles and Practice of Immunoassays Stockton Press, NY; and Ngo (ed.) (1988) Non isotopic Immunoassays Plenum Press, NY.

An HIV transcript, antibody or polypeptide is preferably quantified in a biological sample, such as a cell, or a tissue sample derived from a patient. In a preferred embodiment, antisera to HIV polypeptides are quantified in serum. In another preferred embodiment, HIV nucleic acids are detected in an infected patient using gene probes derived from the nucleic acids of the invention. For instance, in one embodiment, HIV nucleic acids in a biological sample are amplified by an in vitro amplification technique (e.g., PCR or LCR) and detected using labeled complementary nucleic acids.

The sample is pretreated as necessary by dilution in an appropriate buffer solution, or concentrated, if desired. Many standard aqueous buffer solutions employing one of a variety of buffers, such as phosphate, Tris, or the like, at physiological pH are appropriate. Cell sorting techniques such as FACS are optionally used to isolate particular cells such as CD4⁺ cells in which the virus needs to be quantitated.

HIV antibodies, polypeptides and nucleic acids of the invention are detected and quantified by any of a number of means well known to those of skill in the art. These include analytic biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like. The detection of nucleic acids proceeds by well known methods such as Southern analysis, northern analysis, gel electrophoresis, PCR, radiolabeling, scintillation counting, and affinity chromatography.

One of skill will appreciate that it is often desirable to reduce non specific binding in immunoassays or nucleic acid assays, and during analyte purification. Where the assay involves a viral antibody, or other capture agent immobilized on a solid substrate, it is desirable to minimize the amount of non specific binding to the substrate. Means of reducing such non specific binding are well known to those of skill in the art. Typically, this involves coating the substrate with a proteinaceous composition. In particular, protein compositions such as bovine serum albumin (BSA), nonfat powdered milk, and gelatin are widely used.

Western blot analysis can also be used to detect and quantify the presence of a polypeptide or antibody (including peptide, transcript, or enzymatic digestion product) in the sample. The technique generally comprises separating sample products by gel electrophoresis on the basis of molecular weight, transferring the separated proteins to a suitable solid support, (such as a nitrocellulose filter, a nylon filter, or derivatized nylon filter), and incubating the sample with labeling antibodies that specifically bind to the analyte protein (antibody or HIV-2 polypeptide). The labeling antibodies specifically bind to analyte on the solid support. These antibodies are directly labeled, or alternatively are subsequently detected using labeling agents such as antibodies (e.g., labeled sheep anti-mouse antibodies where the antibody to an analyte is a murine antibody) that specifically bind to the labeling antibody.

Other assay formats include liposome immunoassays (LIAs), which use liposomes designed to bind specific molecules (e.g., antibodies) and release encapsulated reagents or markers. The released chemicals are then detected according to standard techniques (see, Monroe et al., (1986) Amer. Clin. Prod. Rev. 5:34-41).

### G. Identification of Modulators of Polypeptides of the Invention

Modulators of a polypeptide of the invention, i.e. agonists or antagonists of a polypeptide's activity, or polypeptide's or polynucleotide's expression, are useful for treating disorders related to HIV infection, as described herein. For example, administration of modulators can be used to treat AIDS patients or HIV infected individuals to prevent progression, and therefore symptoms, associated with HIV or AIDS.

Preferred modulators of the invention are those that act to increase defensin activity at the protein level, e.g., by increasing the half life of defensin, by providing enhanced processing of preprodefensin molecules, more efficient or enhanced translation, by sequestering cellular inhibitors of defensin, by preventing degradation of defensin, increasing secretion and transport of defensin, and providing important molecules in the defensin pathway. Preferred modulators include those that increase expression of defensin at the nucleic acid level, e.g., activators of the defensin promoter, compounds that increase chromosome accessibility of the defensin gene, compounds that increase defensin RNA stability and processing, and compounds that increase defensin RNA levels in the cytoplasm or nucleus.

### 1. Agents that Modulate Polypeptides of the Invention

The compounds tested as modulators of defensin proteins can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of a defensin protein. Typically, test compounds will be small organic molecules, peptides, RNA, antisense molecules, ribozymes, and lipids.

Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

### 2. Methods of Screening for Modulators of the Polypeptides of the Invention

A number of different in vitro and in vivo screening protocols can be utilized to identify agents that modulate the level of expression or activity of a polynucleotide of a polypeptide of the invention in cells, particularly mammalian cells, and especially human cells. In general terms, the screening methods involve screening a plurality of agents to identify an agent that modulates the activity of a polypeptide of the invention by, e.g., binding to the polypeptide, preventing an inhibitor or activator from binding to the polypeptide, increasing association of an inhibitor or activator with the polypeptide, or activating or inhibiting expression of the polypeptide.

Any cell expressing a full-length polypeptide of the invention or a fragment thereof can be used to identify modulators. In some embodiments, the cells are eukaryotic cells lines (e.g., CHO) transformed to express a heterologous polypeptide of the invention. In some embodiments, a cell (e.g., CD4) expressing an endogenous polypeptide of the invention is used in screens. In other embodiments, modulators are screened for their ability to inhibit HIV replication.

### a) Polypeptide Binding Assays

Preliminary screens can be conducted by screening for agents capable of binding to polypeptides of the invention, as at least some of the agents so identified are likely modulators of a polypeptide of the invention. Binding assays are also useful, e.g., for identifying endogenous proteins that interact with polypeptides of the invention. For example, antibodies, receptors or other molecules that bind polypeptides of the invention can be identified in binding assays.

Binding assays usually involve contacting a polypeptide of the invention with one or more test agents and allowing sufficient time for the protein and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound to a polypeptide of the invention or displacement of labeled substrates. The polypeptides of the invention utilized in such assays can be naturally expressed, cloned or synthesized.

In one embodiment, a high throughput binding assay is performed to identify molecules that bind to defensin in which the defensin protein or a fragment thereof is contacted with a potential modulator and incubated for a suitable amount of time and binding between the defensin and the molecule is detected and/or quantified. In one embodiment, the potential modulator is bound to a solid support, and the defensin protein is added. In another embodiment, the defensin protein is bound to a solid support. A wide variety of modulators can be used, as described below, including small organic molecules, peptides, antibodies, and defensin ligand analogs. A wide variety of assays can be used to identify defensin-modulator binding, including labeled protein-protein binding assays, electrophoretic mobility shifts, immunoassays, enzymatic assays such as kinase assays, and the like. In some cases, the binding of the candidate modulator is determined through the use of competitive binding assays, where interference with binding of a known ligand or substrate is measured in the presence of a potential modulator. Either the modulator or the known ligand or substrate is bound first, and then the competitor is added. After the defensin protein is washed, interference with binding, either of the potential modulator or of the known ligand or substrate, is determined. Often, either the potential modulator or the known ligand or substrate is labeled. In one embodiment the assay is a SELDI-based assay, such as those described above under the heading “2. SELDI Detection of Defensin Polypeptides.” In such an assay one member of the defensin/test compound pair is bound to the surface of a SELDI-based biochip (e.g., a preactivated ProteinChip array from Ciphergen Biosystems, Inc.) and the other member is contacted with the surface of the biochip. Binding between the pair is detected by SELDI. For example, a defensin polypeptide can be bound to the surface of a biochip and a library of test compounds can be exposed to the bound defensin. Unbound molecules are washed away. Then the bound molecules are detected by SELDI.

High throughput screening assays involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res., 37:487-493 (1991) and Houghton et al., Nature, 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA, 90:6909-6913 (1993)), vinylogous polypeptides (Hagihara et al., J. Amer. Chem. Soc., 114:6568 (1992)), nonpeptidal peptidomimetics with glucose scaffolding (Hirschmann et al., J. Amer. Chem. Soc., 114:9217-9218 (1992)), analogous organic syntheses of small compound libraries (Chen et al., J. Amer. Chem. Soc., 116:2661 (1994)), oligocarbamates (Cho et al., Science, 261:1303 (1993)), and/or peptidyl phosphonates (Campbell et al., J. Org. Chem., 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In addition, mammalian or yeast two-hybrid approaches (see, e.g., Bartel, P. L. et. al. Methods Enzymol, 254:241 (1995)) can be used to identify polypeptides or other molecules that interact or bind when expressed together in a host cell.

### b) Polypeptide Activity

The activity of polypeptides of the invention can be assessed using a variety of in vitro and in vivo assays to determine functional, chemical, and physical effects, e.g., enzymatic activity, cell proliferation (e.g., CD4⁺ lymphocyte proliferation), HIV replication, expression of HIV proteins, or ligand or substrate binding. Furthermore, such assays can be used to test for inhibitors and activators of the polypeptides of the invention. Modulators can also be genetically altered versions of polypeptides of the invention.

The polypeptide of the assay will be selected from a polypeptide with a sequence as encoded by an accession number or SEQ ID NO: 1-24 described herein or a conservatively modified variant thereof. Alternatively, the defensin protein will be derived from a eukaryote and include an amino acid subsequence having substantial amino acid sequence identity to an accession number described herein. Generally, the amino acid sequence identity will be at least 60%, preferably at least 65%, 70%, 75%, 80%, 85%, or 90%, most preferably at least 95%. Optionally, the polypeptide of the assays will comprise a fragment of a polypeptide of the invention, such as an extracellular domain, transmembrane domain, cytoplasmic domain, ligand binding domain, subunit association domain, active site, and the like. Either a polypeptide of the invention or a domain thereof can be covalently linked to a heterologous protein to create a chimeric protein used in the assays described herein.

Modulators of polypeptide activity are tested using either recombinant or naturally occurring polypeptides of the invention. The protein can be isolated, expressed in a cell, expressed in a membrane derived from a cell, expressed in tissue or in an animal, either recombinant or naturally occurring. For example, tissue slices, dissociated cells, e.g., from tissues expressing polypeptides of the invention, transformed cells, or membranes can be used. Modulation is tested using one of the in vitro or in vivo assays described herein.

Modulator binding to polypeptides of the invention, a domain, or chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbance, refractive index), hydrodynamic (e.g., shape), chromatographic, or solubility properties.

Samples or assays that are treated with a potential modulator (e.g., a “test compound”) are compared to control samples without the test compound, to examine the extent of modulation. Control samples (untreated with activators or inhibitors) are assigned a relative activity value of 100. Inhibition of the polypeptides of the invention is achieved when the activity value relative to the control is about 90%, optionally 50%, optionally 25-0%. Activation of the polypeptides of the invention is achieved when the activity value relative to the control is 110%, optionally 150%, 200%, 300%, 400%, 500%, or 1000-2000%.

### c) Expression Assays

Screening for a compound that modulates the expression of a polynucleotide or a polypeptide of the invention is also provided. Screening methods generally involve conducting cell-based assays in which test compounds are contacted with one or more cells expressing a polynucleotide or a polypeptide of the invention, and then detecting an increase or decrease in expression (either transcript or translation product). Assays can be performed with any cells that express a polynucleotide or a polypeptide of the invention. Expression can be detected in a number of different ways. As described infra, the expression level of a polynucleotide of the invention in a cell can be determined by probing the mRNA expressed in a cell with a probe that specifically hybridizes with a transcript (or complementary nucleic acid derived therefrom) of a polynucleotide of the invention. Probing can be conducted by lysing the cells and conducting Northern blots or without lysing the cells using in situ-hybridization techniques. Alternatively, a polypeptide of the invention can be detected using immunological methods in which a cell lysate is probed with antibodies that specifically bind to the polypeptide.

The level of expression or activity of a polynucleotide or a polypeptide of the invention can be compared to a baseline value. The baseline value can be a value for a control sample or a statistical value that is representative of expression levels of a polynucleotide or a polypeptide of the invention for a specific population (e.g., progressors) or cells (e.g., tissue culture cells not exposed to a modulator). Suitable cells for such cell based assays include both primary cells such as PBMCs, lymphocytes (e.g., CD4⁺), neutrophils, polymorphonuclear leukocytes, and other phagocytic cells and cell lines, e.g., Jurkat cells, BJAB cells, etc. The defensin protein can be naturally occurring, chemically synthesized or recombinant.

### d) Animal Models

Animal models of HIV infection also find use in screening for modulators of defensins. Similarly, transgenic animal technology including gene knockout technology, for example as a result of homologous recombination with an appropriate gene targeting vector, or gene overexpression, will result in the absence or increased expression of the defensin protein. The same technology can also be applied to make knock-out cells.

Knock-out cells and transgenic mice can be made by insertion of a marker gene or other heterologous gene into an endogenous defensin gene site in the mouse genome via homologous recombination. Such mice can also be made by substituting an endogenous defensin with a mutated version of the defensin gene, or by mutating an endogenous defensin, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice, it is possible to obtain a new line of mice containing the introduced genetic lesion (see, e.g., Capecchi et al., Science, 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

### e) Solid Phase and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 or more different compounds are possible using the integrated systems of the invention. In addition, microfluidic approaches to reagent manipulation can be used.

A molecule of interest (e.g., a polypeptide or polynucleotide of the invention, or a modulator thereof) can be bound to the solid-state component, directly or indirectly, via covalent or non-covalent linkage, e.g., via a tag. The tag can be any of a variety of components. In general, a molecule that binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fe region of an immunoglobulin, poly-His, etc.) Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody that recognizes the first antibody. In addition to antibody-antigen interactions, receptor-ligand interactions are also appropriate as tag and tag-binder pairs, such as agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to those of skill in the art. For example, poly(ethelyne glycol) linkers are available from Shearwater Polymers, Inc., Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent that fixes a chemical group to the surface that is reactive with a portion of the tag binder. For example, groups that are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer 10 arrays is well described in the literature (see, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank and Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

The invention provides in vitro assays for identifying, in a high throughput format, compounds that can modulate the expression or activity of a polypeptide of the invention, e.g., immunoassays such as ELISA. Control reactions that measure activity of a polypeptide of the invention in a cell in a reaction that does not include a potential modulator are optional, as the assays are highly uniform. Such optional control reactions are appropriate and increase the reliability of the assay. Accordingly, in some embodiments, the methods of the invention include such a control reaction. For each of the assay formats described, “no modulator” control reactions that do not include a modulator provide a background level of binding activity.

In some assays it will be desirable to have positive controls. At least two types of positive controls are appropriate. First, a known activator of a polypeptide or a polynucleotide of the invention can be incubated with one sample of the assay, and the resulting increase in signal resulting from an increased expression level or activity of a polypeptide or a polynucleotide of the invention are determined according to the methods herein. Second, a known inhibitor of a polypeptide or a polynucleotide of the invention can be added, and the resulting decrease in signal for the expression or activity of a polypeptide or a polynucleotide of the invention can be similarly detected. It will be appreciated that modulators can also be combined with activators or inhibitors to find modulators that inhibit the increase or decrease that is otherwise caused by the presence of the known modulator of a polypeptide or a polynucleotide of the invention.

### H. Assays for Molecules that Interact with Defensin Polypeptides

In addition to providing assays that can be used to identify modulators of defensin polypeptides, the present invention also provides assays for molecules that interact with defensin polypeptides. Molecules from CD4⁺ cells that interact with alpha-defensin may be important in the anti-viral activity of alpha-defensin and, therefore, may be suitable targets for pharmaceutical intervention. Therefore, this invention provides assays for detecting such molecules. The methods involve exposing CD4⁺ cells with alpha-defensin for sufficient time to induce the antiviral response, and then capturing molecules that bind alpha-defensin from the CD4⁺ cells or from the culture medium in which the cells are cultured. Capturing molecules that bind to alpha-defensin can be done indirectly or directly.

In a direct method, after exposure to alpha-defensin, material from the cell is exposed to an immobilized alpha-defensin, e.g., a derivatized bead or an affinity biochip, e.g., a ProteinChip array. Any molecules that bind alpha-defensin will be captured on the solid phase. Then, the solid phase can be washed to remove unbound material and the molecules that have bound to alpha-defensin can be eluted and detected by any of the many means known for detection.

In an indirect method, after exposure to alpha-defensin, material from the cell is exposed to a capture agent that binds alpha-defensin. This could be, for example, an immobilized antibody against alpha-defensin or an antibody against alpha-defensin in solution that can itself then be captured on a solid phase. In this way, any molecules bound to alpha-defensin will be captured along with the alpha-defensin. Then, the solid phase can be washed to remove unbound material and the molecules that have bound to alpha-defensin can be eluted and detected.

### I. Kits

This invention also provides kits for detecting defensins. Such kits are useful, for example, for diagnostic or prognostic tests. Kits generally will include a solid phase for capturing the defensin and adapted for detection of captured molecules on the solid phase. Accordingly, such solid phases can be derivatized with an antibody against a defensin or the kit can include an antibody with which the substrate will be derivatized. Such solid phases include microtiter plates, beads, or biochips adapted for mass spectrometry, e.g., ProteinChip arrays available from Ciphergen Biosystems, Inc. (e.g., PS1 ProteinChip array or PS2 ProteinChip array). Particularly useful are kits that comprise capture reagents or detection reagents for at least two different defensins or at least three different defensins. In the case of the alpha-defensins, a single antibody can capture alpha-defensin 1, 2 and 3 due to their common sequences. Distinguishing these alpha-defensins can be accomplished by mass spectrometry, in they can be differentiated by mass, or by sandwich immunoassay, by using antibodies specifically directed to the amino-terminal end of the alpha-defensin. The kit can also include instructions to detect and quantify the defensins in a sample, as well as instructions to correlate the amount detected with a prognosis or HIV infection status.

### EXAMPLES

The following examples are offered to illustrate, but not to limit the claimed invention.

### Example I

### Materials and Methods

Preparation of CD8⁺ T cells and their stimulated culture supernatants. Venous blood samples were collected from two long-term survivors, who have remained healthy despite infection with HIV-1 for over 13 years (CD4 and viral load). Additional blood samples were also collected from 4 HIV-1 infected progressors prior to anti-viral therapy (CD4 and viral load), and from 15 uninfected normal individuals. Peripheral blood mononuclear cells (PBMC) were then separated by standard Ficoll/Hypaque gradient centrifugation. The CD8⁺ T cells were isolated from PBMC by positive selection using magnetic beads coated with anti-CD8 monoclonal antibody (Dynal, Lake Success, N.Y.). Beads were then removed from the cells by Detach-a-Bead (Dynal). The purity of the cells obtained by this procedure is >99.5%, judged by FACS. Isolated CD8⁺ T cells were stimulated for 3 days at 37° C. in a humidified CO₂ incubator with 0.1 μg/ml anti-CD3 (12F6) (gift from Johnson at MIT) and 5 μl/ml anti-CD28 (Becton Dickinson) antibodies together with equal number of allogeneic irradiated PBMC in serum-free RPMI 1640 medium (Cellgro), supplemented with recombinant IL-2 (20 U/ml), penicillin (100 U/ml) and streptomycin (100 μg/ml). Culture supernatants were collected and cleared by brief centrifugation at 3000 rpm for 5 min, and stored at −80° C. until use.

ProteinChip® Array analysis of cell culture supernatants. Each ProteinChip Array has distinct chemical (ionic, hydrophobic, hydrophilic, etc.) or biological (antibody, receptor, DNA, etc) characteristics designed for selective capture of proteins/peptides from complex mixtures. Multiple types of ProteinChip arrays were used initially in our experiment including weak cation exchanger (WCX2), strong anion exchanger (SAX2), immobilized metal affinity capture (IMAC-3) and reverse phase H4 (Ciphergen). The WCX2 array was eventually chosen for the entire study due to its high reproducibility in our hands in detecting proteins/peptides species from the culture supernatant. Specifically, 100 μl of culture supernatant was mixed with an equal volume of binding buffer (100 mM NaAc, pH 4.5; 0.2% Triton X-100 in PBS). The mixture was then applied to the WCX2 array through a bioprocessor (Ciphergen) and incubated at 4° C. overnight with constant horizontal shaking. Unbound proteins/peptides were removed by washing with the binding buffer (50 mM NaAc. PH 4.5; 0.1% Triton X-100 in PBS) three times for 5 min each under the same temperature and shaking conditions. The WCX2 arrays were then disassembled from the bioprocessor, rinsed in 1 mM HEPES (pH 4.5) for 30 sec, and air-dried. After drying, a saturated solution of 3,5-Dimethoxy-4-hydroxycinnamic acid (SPA) in 50% acetonitrile and 0.5% trifluoroacetic acid was added to the chip surface. SPA serves as an energy-absorbing molecule for the captured proteins/peptides which are detected by the surface-enhanced laser desorption/ionization (SELDI) time-of-flight mass spectrometry (Ciphergen). The arrays were then placed in the Ciphergen Protein Biological System II (PBS II), where nanosecond laser pulses are generated and fired from a nitrogen laser (337 nm) at the mixture of SPA and proteins/peptides. The mass spectra of proteins/peptides were generated using an average of 80 laser shots at laser intensity of 220-240. The mass to charge ratio (M/Z) of each of the proteins/peptides captured on the array surface was determined according to the external proteins/peptides standard calibrated by ARG-8-Vasopressin (1,084,3 Da), Somatostatin (1,637.9 Da), Bovine insulin β chain (3,495.9 Da), Human insulin (5,807.7), and Hirudin (7,033.6 Da) (Ciphergen).

Protein identification using antibody-coated beads followed by SELDI ProteinChip® analysis. Dynal beads were coated with human neutrophil (alpha) defensin-specific antibody or human macrophage inflammatory protein (MIP) 1 alpha-specific antibody according to manufacturer's instructions. In brief, biotinylated antibody specific for human neutrophil defensins 1-3 (clone D21, HyCult biotechnology) was mixed with Biotin-Binder Dynabeads (Dynal) in PBS containing 0. 1% Tween 20, and rotated at room temperature for 30 min. Unbound antibody was removed by washing with PBS containing 0.1% Tween 20 for three times. In addition, human MIP-1 alpha-specific antibody (R&D systems) was directly coupled to the Dynabeads M-450 Epoxy (Dynal) in 0.1 M Borate buffer (pH 7.0-7.5) by rotating at 4° C. overnight. After 15-30 min incubation, 0.5% BSA was added to ensure an optimal orientation of the antibody. Antibody-coated beads were then washed with PBS containing 0.1% BSA (pH 7.4) three times. For every 10⁷ beads, approximately 2-5 μg of each antibody is required. The antibody-coated beads (approximately 4×10⁷) were then mixed individually with 200 μl of culture supernatants from stimulated CD8⁺ T cells in the presence of 0.01% of hexadecyltrimethylammonium bromide (CETAB, Sigma, St. Louis, Mo.), and rotated at 4° C. overnight. The antigen-antibody complexes-coated beads were then removed from the supernatant by a magnet (Dynal), and the remaining supernatants were applied to the WCX2 arrays and analyzed by SELDI as illustrated above.

Preparation of enriched and depleted cell culture supernatants from stimulated CD8⁺ T cells for anti-viral assays and protein identification by Qq-TOF. A BioSepra Q-HyperD column (Ciphergen) was equilibrated and washed with 50 mM Tris HCl (pH 8.0) containing 10% acetonitrile (CAN) and 0.1% trifluoroacetic acid (TFA). To enrich the putative alpha-defensin peptides, the culture supernatant was mixed with 50% acetonitrile in 50 mM Tris HCl (pH 8.0) and applied to the column. Flow through was collected, diluted five-fold in the same buffer and applied directly to a BioSepra Reverse Phase C18 column. The bound proteins/peptides were eluted using 50 mM Tris HCl (pH 8.0) with 0.1% TFA and an escalating amount of ACN in the solution from ranging from 30%, 40%, 50%, 60%, and finally 80%.

Protein identification using Micromass Q-TOF II. The eluted fraction from the BioSepra Reverse Phase C18 column containing 50% ACN and 0.1% TFA. A neutralization of the TFA was performed by adding 10% v/v 1 OOmM ammonium bicarbonate buffer (pH 8) and then the neutralized eluate was concentrated via speed vacuum at room temperature for 30 min until the solution was reached approximately 25 femtomoles proteins/peptides per microliter. The concentrated material was then reduced with 5 mM DTT in 50 mM Tris HCl (pH 8.6-9) for 5 min at 90° C., but no alkylation was performed. The reduced mixture was cooled to room temperature and was digested with 0.6 μg/μl trypsin at 40° C. for 2 h and then stopped on ice. All of the above reactions were performed under inert argon gas. Trypsin-digested products were then directly applied to a Ciphergen NP-20 array, and protein identity was determined by the Micromass Q-TOF II through the Ciphergen PCI-1000 ProteinChip® Interface.

Virus inhibition assays. Virus inhibition assays were performed by using both pseudotyped single-cycle viruses and replication-competent HIV-1 strains isolated from HIV-1 infected patients on either CEM 174 cell line and/or PBMC. CEM 174 cell line has been engineered to expression both HIV-1 co-receptors CXCR4 and CCR5. Pseudotyped viruses were generated by co-transfection of the luciferase-reporter virus backbone pNL4-3 LucR⁻E⁻ with an envelope expression vector for either JR-FL or HXB2 into the human embryonic kidney cell line 293T. The virus-containing supernatants were collected 2 days later, and p24 antigen levels in the supernatants were quantified using standard protocol (ref). The supernatants were then aliquoted and stored at −80° C. until use. For virus inhibition assay using pseudotyped single-cycle virus, 2×10⁵ CEM 174 cells were seeded per well in a 96-well plate the day prior to infection. Five nanogram of p24 single-cycle JR-FL or HXB2 reporter virus was added to each well in the presence of either human neutrophil defensin 1 or 2 (American Peptide Company, Inc. Sunnyvale, Calif.) singly or in combination. To study the possible mechanism of inhibition, human neutrophil defensins or culture supernatants from stimulated LTNP's CD8⁺ T cells were added either simultaneously with the virus or at timed intervals after infection. A viral entry inhibitor T-20 and a non-nucleotide reverse transcriptase inhibitor UC-781 were also included in the experiment for assessing the timing of each respective step during the viral life cycle. Three days after initial infection, cells were washed with PBS, disrupted in 50 μl luciferase lysis buffer (Promega), and frozen-thawed once to increase the detection sensitivity. After a brief centrifugation step at 3,000 rpm for 10 min, 25 μl of cell lysates were measured for luciferase activity using the Promega Luciferase Assay System and a luminometer (Dynex Technologic, Inc.).

For virus inhibition assay using replication-competent viruses, PBMC was firstly stimulated with PHA at the concentration of 20 μg/ml for 3 days. Approximately 2×10⁵ cells were seeded per well in a 96-well plate with 100 tissue culture infectious dose (TCID₅₀) of each virus in the presence of either human neutrophil defensin 1 or 2 singly or in combination. In some experiments, culture supernatants from stimulated LTNP's CD8⁺ T cells were also included. After infection for 2 h at 37° C., the remaining viruses in the supernatant were removed by extensive washing with PBS. Fresh medium containing the same concentration of human neutrophil defensin or culture supernatant from stimulated LTNP's CD8⁺ T cells were added. The p24 antigen levels in the supernatants were measured on day 0, 3 and 7 post-infection using a standard protocol. The percent inhibition was determined by comparing the p24 levels in the supernatant with or without inhibitors at the time of peak virus production, normally on day 7 after initial infection.

### Results

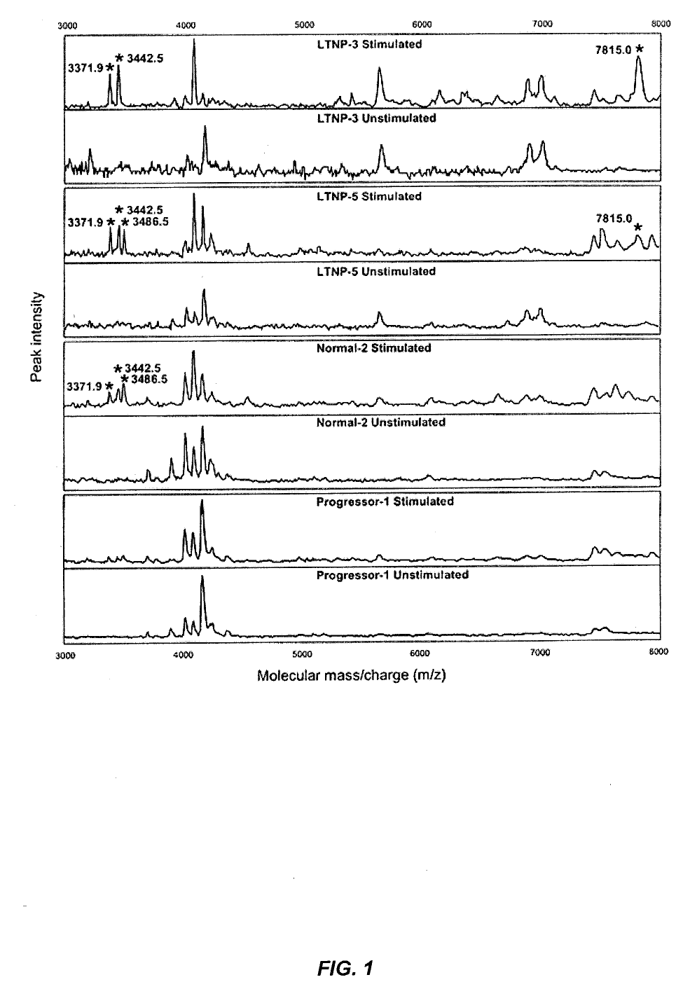

Identification of a cluster of small proteins secreted by CD8⁺ cells from LTNP and normal persons. Supernatant fluids were harvested from stimulated and unstimulated CD8 T-lymphocyte cultures derived from 3 LTNP, as well as from 4 infected progressors and 15 normal controls. Each sample was analyzed on the ProteinChip® System (Ciphergen Biosystems, Inc., Fremont, Calif.), which is based on the integration of chemically modified array surfaces with surface-enhanced laser desorption/ionization (SELDI) time-of-flight (TOF) mass-spectrometry (MS) detection. This technology was chosen because of its power of resolution, high reproducibility, ease of use, and femtomole level sensitivity. As shown in FIG. 1a, representative protein mass spectra for two LTNP and one normal control revealed significant differences in peak pattern between stimulated and unstimulated CD8 supernatants. A cluster of two or three peaks, with m.w. of 3,371.9 Da, 3,442.5 Da, and 3,486.5 Da, was found in stimulated cultures. This cluster of molecules was detected in cultures of stimulated CD8 T-lymphocytes from 3 of 3 LNTP and 11 of 15 normal individuals, but not in any stimulated culture from 4 progressors (FIG. 1a). A unique peak at 7,815.0 Da, later identified as MIP-1α, was also detected in stimulated samples from two LTNP. Although plenty of peaks were observed from 8,000 to 200,000 Da, no significant differences were found between stimulated and unstimulated CD8 cultures for the three study groups (data not shown). In particular, no peak greater than 8,000 Da consistently correlated with the presence of CAF activity.

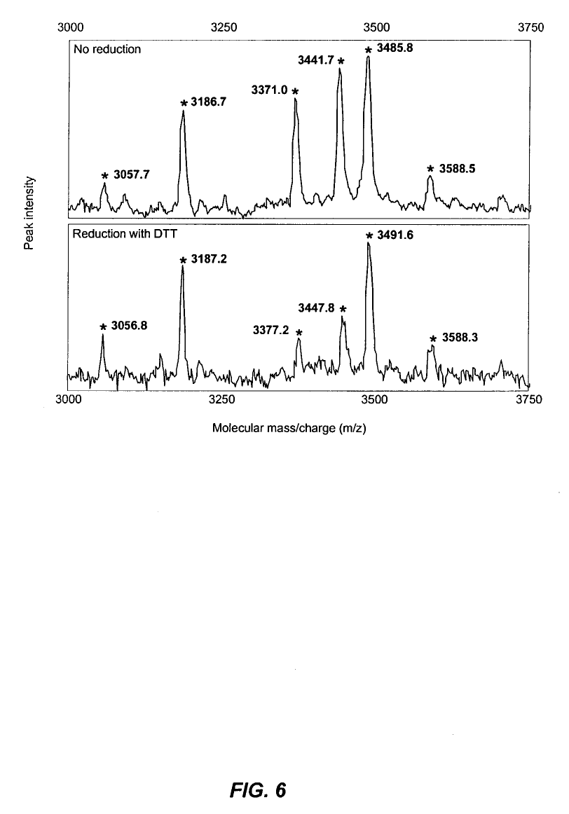

To further characterize the cluster of peaks between 3,300 and 3,500 Da, culture supernatants from stimulated CD8 T cells from LTNP subject-3 (LTNP-3) and normal control number-2 (Normal-2) were enriched for these proteins as described. Enriched materials were then treated with dithiothreitol (DTT), acrylamide, or iodoacetamide to probe for existence of disulfide bonds within each protein in the cluster. The resultant materials were then directly applied onto Ciphergen NP-20 arrays and assayed by SELDI-TOF-MS. Table 1 below shows the changes in molecular mass for the three peaks found in Normal-2 upon reduction with DTT. Each peak gained ˜6 Da after reduction (FIG. 6), suggesting that every protein in the cluster contains three internal disulfide bridges, because DTT reduction would add two hydrogen atoms to form two free sulfhydryl groups for each disulfide bond broken. Furthermore, for peaks detected in the culture supernatant from LTNP-3, reduction and alkylation with acrylamide or iodoacetamide led to increases of ˜434 Da or 349 Da, respectively. Given the molecular weights of acrylamide (m.w. 71) and iodoacetamide (m.w. 57), the observed mass increases were consistent with the addition of 6 acrylamide or iodoacetamide molecules to each protein through six free sulfhydryl groups. This result further confirmed the presence of three intramolecular disulfide bridges in each protein in the cluster.

TABLE 1
Changes in molecular mass (m/z) before [−] and after [+] reduction ± alkylation.

| | dithiothreitol | | | | acrylamide | | | iodoacetamide | | |
|---|---|---|---|---|---|---|---|---|---|---|
| | | | net | | | | net | | | net |
| Normal-2 | [−] | [+] | change | LTNP-3 | [−] | [+] | change | [−] | [+] | change |
| peak 1 | 3371.0 | 3377.2 | 6.2 | peak 1 | 3370.0 | 3804.3 | 434.3 | 3371.5 | 3720.0 | 348.5 |
| peak 2 | 3441.7 | 3447.8 | 6.1 | peak 2 | 3441.3 | 3875.0 | 433.7 | 3442.1 | 3791.3 | 349.2 |
| peak 3 | 3485.8 | 3491.6 | 5.8 | peak 3 | n/d | n/d | n/d | n/d | n/d | n/d |

| | | | |
|---|---|---|---|
| Average | 6.0 | 434.0 | 348.9 |
| # of disulfide bonds | 3 | 3 | 3 |

n/d: not detected

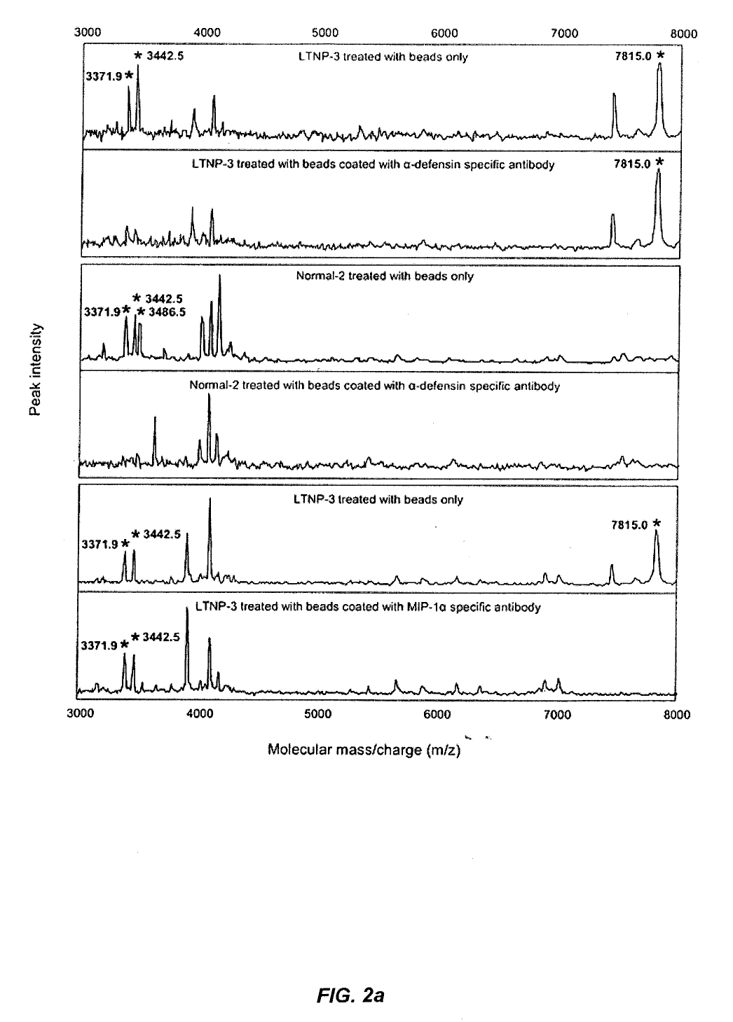

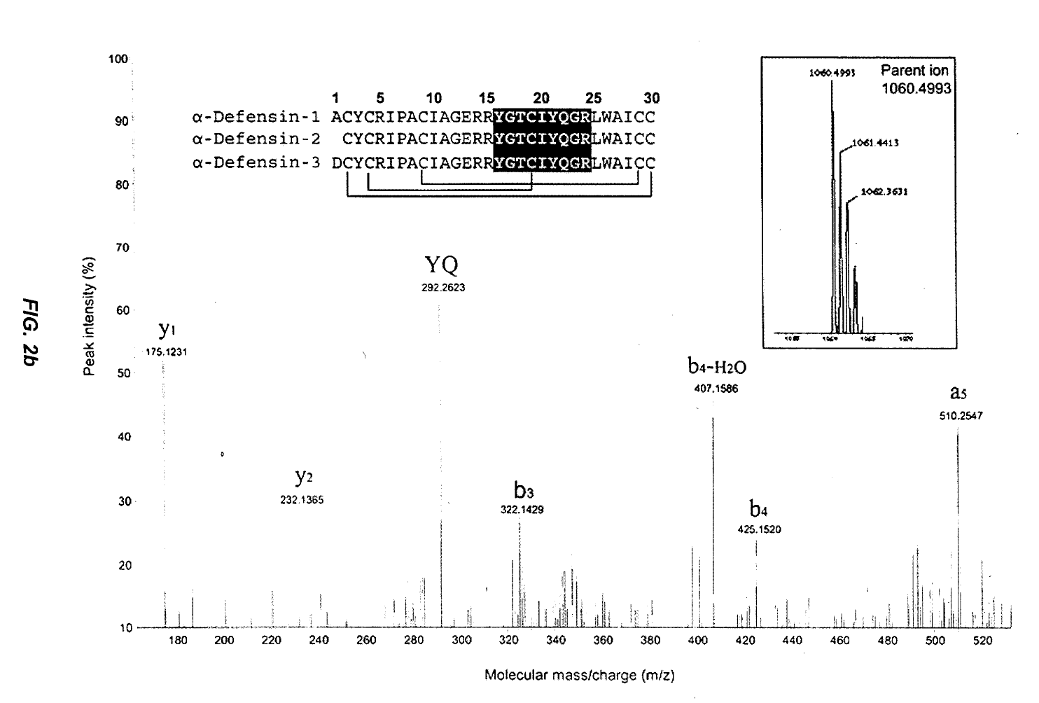

Identification of the protein clusters as human alpha-defensins 1, 2, and 3. By searching through the protein databases (NCBI and Swiss-Pro) for proteins with similar molecular weight to the peaks up-regulated after stimulation, it was found that the doublet or triplet peaks with an average mass of 3,371.9 Da, 3,442.5 Da, and 3,486.5 Da correspond to human neutrophil (alpha) defensins 2, 1 and 3, respectively, which are peptide antibiotics made principally by human neutrophils (Ganz et al., J. Clin. Invest., 76:1427 (1985), Selsted et al., J. Clin. Invest., 76:1436 (1985), Daher et al., Proc. Natl. Acad. Sci. USA, 85:7327 (1988))). In addition, each of these peptides is known to contain three internal disulfide bonds (Lehrer et al., Curr. Opin. Immun. 14, 96 (2002)). The 7,815.0 Da peak corresponds to human MIP-1 alpha. In addition, it has been previously demonstrated that approximately 10% of the population lacks defensin 3 (Mars et al., J. Biol. Chem., 270:30371 (1995), which helps to explain why some study subjects display a two-peak cluster whereas others have a three-peak cluster in both LTNP and normal individuals (FIG. 1). To test this hypothesis, Dynal beads coated with antibody specific for human alpha-defensin 1, 2, and 3 were incubated with supernatant from stimulated CD8⁺ T cells, and then removed by magnet. In brief, biotinylated monoclonal antibody specific for human α-defensins-1,2,3 (clone D21, HyCult Biotechnology, Norwood, Mass.) was mixed with Biotin-Binder Dynabeads (Dynal) in PBS containing 0.1% Tween 20, and rotated at room temperature for 30 min. Unbound antibodies were removed by washing three times with PBS containing 0.1% Tween 20. Separately, human-MIP-1α-specific monoclonal antibody (R&D Systems, Minneapolis, Minn.) was directly coupled to Dynabeads M-450 Epoxy (Dynal) in 0.1 M borate buffer (pH 7.0-7.5) by rotating at 4° C. overnight. After 15-30 min of incubation, 0.5% BSA was added to ensure an optimal orientation of the antibody. Antibody-coated beads were then washed three times with PBS containing 0.1% BSA (pH 7.4). For every 10⁷ beads, approximately 2-5 μg of each antibody was used. The antibody-coated beads (approximately 4×10⁷) were then mixed individually with 200 μl of culture supernatants from stimulated CD8⁺ T cells in the presence of 0.01% of hexadecyltrimethylammonium bromide (Sigma, St. Louis, Mo.), and rotated at 4° C. overnight. The antigen-antibody complexes-coated beads were then removed from the supernatant by a Detach-a-Bead magnet (Dynal), and the remaining supernatants were applied onto WCX2 arrays and analyzed by SELDI-TOF-MS as described above. It was reasoned that should the doublet or triplet peaks indeed belong to the human alpha-defensin family, by incubating with and finally removing those antibody-coated beads from the supernatants would simultaneously deplete the two- or three-peak clusters from the supernatant. Pretreatment with an anti-defensin-1,2,3 antibody eliminated the cluster of proteins in the range of 3,300 to 3,500 Da (FIG. 2a), without significantly affecting other peaks. Preincubation with an anti-MIP-1α antibody did not affect the peaks of interest, but did result in the removal of a peak at 7815.0 Da. These experiments clearly demonstrate that the two- and three-peak protein clusters up-regulated after stimulation of CD8⁺ T cells from LTNP and normal individuals are members of human alpha-defensin family. Beta-chemokine MIP-1 alpha is also up-regulated after stimulation, which is consistent with previous reports.

Protein sequencing of the low MW cluster of protein peaks using Micromass Qq-TOF MS-MS. To further confirm this conclusion, the enriched materials previously used for the reduction and alkylation experiments were digested with trypsin and analyzed by tandem mass spectrometry. Trypsin-digested materials from both LTNP-3 and Normal-2 yielded a unique 1060.50 Da (FIG. 2b, upper-right inset) fragment compared to controls that did not contain the protein cluster of interest. This unique fragment was further selected and fragmented into smaller ions by collision-induced dissociation in the MS-MS collision cell. The seven unique ions generated by this means (FIG. 2b) were then used in a protein search engine (see Legend to FIG. 2b) to look for theoretical fragments of the 1060.50 Da parent ion. The search yielded a perfect and unequivocal match with a trypsin-digest fragment of human α-defensins-1, -2, and -3. In fact, this peptide is conserved among these three molecules and corresponds exactly to the sequence YGTCIYQGR from amino-acid positions 16 to 24 (FIG. 2b). Protein sequencing confirmed that the protein cluster up-regulated after stimulation of CD8 T cells from a LTNP and a normal control are members of the human α-defensin family.

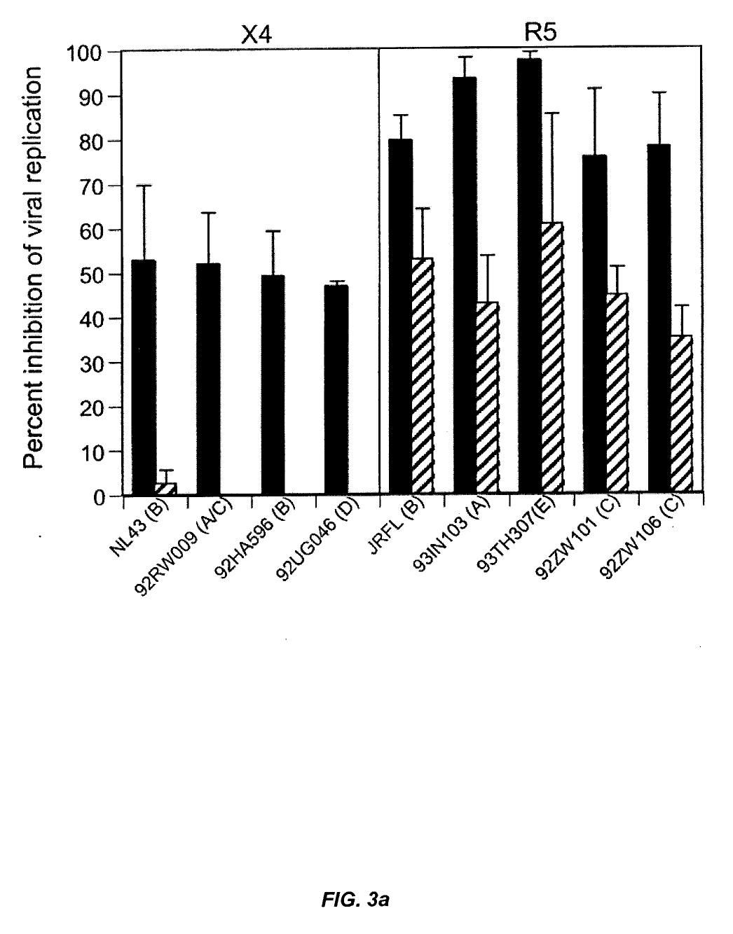

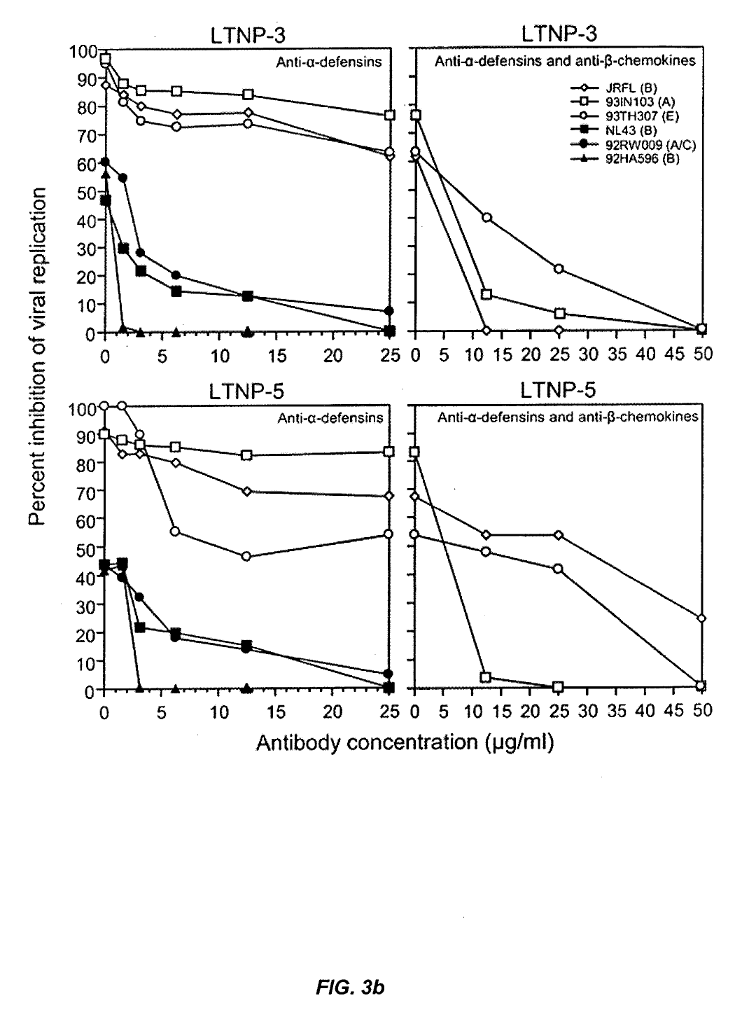

Human α-defensins-1, -2, and -3 account for the HIV-1 suppressive activity of CAF that is not attributable to β chemokines. To evaluate the relative contribution of α-defensins-1, -2, and -3 to the total CAF activity, culture supernatants from stimulated CD8⁺ T cells from LTNP-3 and LTNP-5 were selectively depleted of these molecules using beads coated with a specific antibody as described above or an affinity column (the biotinylated monoclonal antibody from clone D21 (HyCult Biotechnology) was used to deplete α-defensins-1, -2 and -3 from culture supernatants using HiTrap affinity columns according to manufacture's instruction (Amersham Pharmacia Biotech, Piscataway, N.J.)). FIG. 3a compares the antiviral activity (average of two experiments), before and after depletion of α-defensins-1, -2, and -3, against a panel of X4 and R5 HIV-1's, including strains from various genotypes. Viral inhibition assays were performed as described above. Before depletion, culture supernatants were able to inhibit ˜50-60% of the replication of all X4 viruses tested, irrespective of genotype. After depletion, however, the inhibitory effect against X4 viruses was completely eliminated, indicating that α-defensins-1, -2, and -3 account for most, if not all, of the suppressive activity of CAF against X4 viruses. For R5 viruses, there was an average of ˜40% reduction in anti-HV-1 activity after the removal of α-defensins (FIG. 3a).

It was also found that α-defensins 1 and 2 can inhibition replication approximately 15-30% of both viruses at the concentration of 10 μM. However, when similar concentrations of α-defensins 1 and 2 were added together, the percent inhibition increased up to 70%, suggesting a synergistic effect of between these two α-defensins. Furthermore, inhibition of both viruses by α-defensins was dose-dependent.

It was next examined whether CAF activity could be neutralized in a dose-dependent manner by the addition of an anti-α-defensins-1,2,3 antibody to the culture supernatant, with and without the co-addition of antibodies to β-chemokines. Anti-HIV-1 activity of CD8 supernatants from LTNP-3 and LTNP-5 decreased as the concentration of an anti-α-defensin antibody increased (FIG. 3b). For all X4 viruses tested, the suppressive activity of CAF was virtually eliminated when antibody concentration reached 25 μg/ml, while a similar amount of a control antibody had no effect (data not shown). The inhibitory activity against R5 viruses was also reduced by the addition of increasing amounts of an α-defensin antibody, although the effect was not as profound as that observed for X4 viruses (FIG. 3b). To address the possibility that the residual activity against R5 viruses could be due to β-chemokines, culture supernatants from LTNP-3 and LTNP-5 were treated with increasing amounts of a mixture (1:1:1) of antibodies against MIP-1α, MIP-1β and RANTES, together with a fixed concentration (25 μg/ml) of an α-defensin antibody. The residual antiviral activity against three R5 isolates was almost completely neutralized at the highest antibody concentration used (FIG. 3b, right panels), while the addition of a control antibody had minimal effects (data not shown). Collectively, these results demonstrate that α-defensins-1, -2, and -3 account for nearly all of the anti-HIV-1 activity in supernatants of stimulated CD8⁺ T-lymphocyte cultures that is not attributable to β-chemokines.

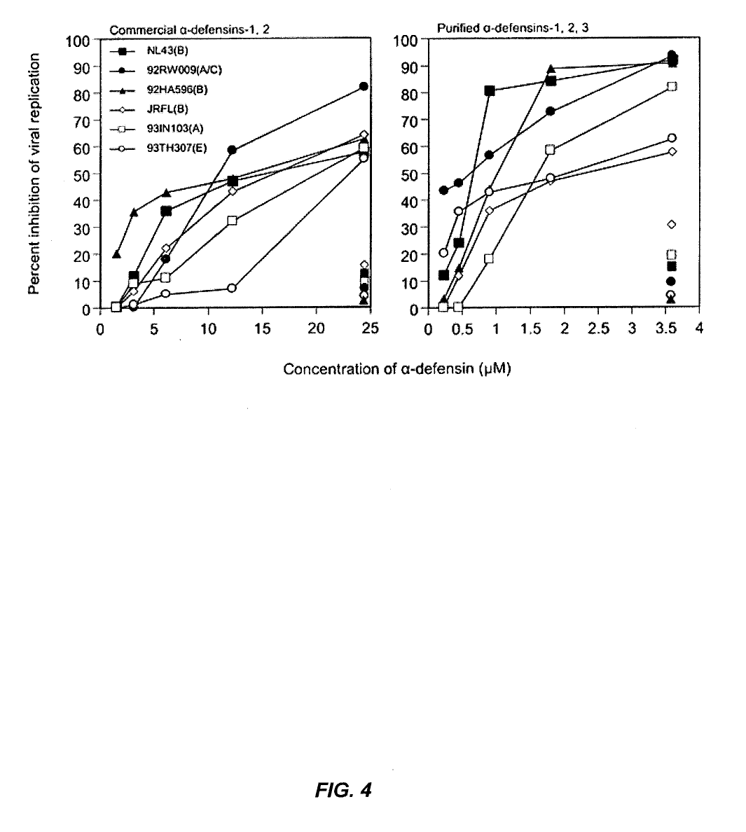

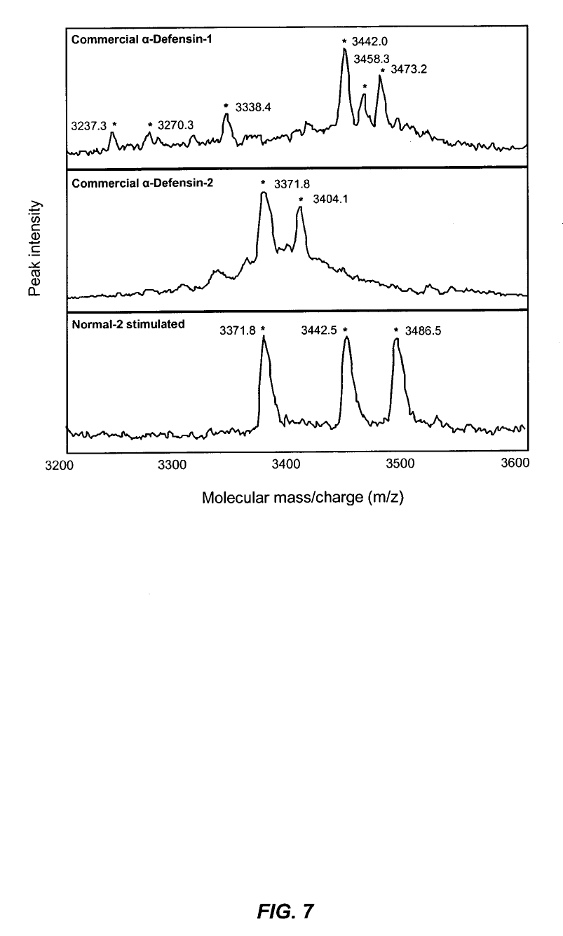

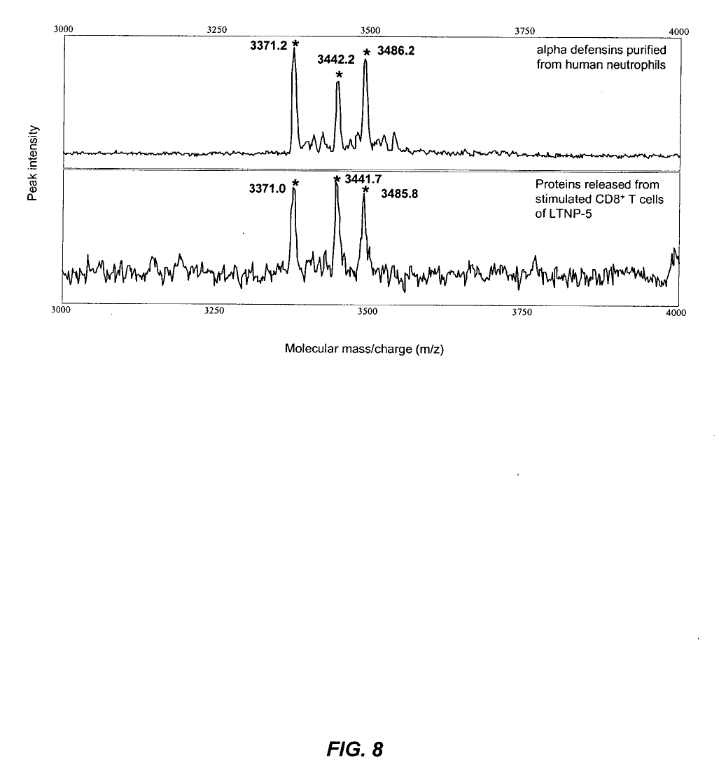

Synthetic and purified human alpha-defensins can inhibit HIV-1 replication in vitro. We next turned our attention to the testing of synthetic or purified forms of α-defensins for anti-HIV-1 activity in vitro. Two products are commercially available: α-defensin-1 and -2 (American Peptide Company, Sunnyvale, Calif.). With increasing concentrations of a mixture (1:1) of these two synthetic α-defensins, a greater degree of inhibition was observed against 6 isolates of HIV-1, (FIG. 4) regardless of viral phenotype or genotype. The 50% inhibitory concentrations (IC₅₀) for the mixture ranged from about 11 1 to 24 μM. While the antiviral potency of this mixture was not great, it should be noted that these commercial products were not pure, as is seen by SELDI-TOF-MS (FIG. 7). Compared to α-defensins produced stimulated CD8⁺ T cells, the commercial products contained several protein peaks other than ones with the right molecular masses. Furthermore, even for the peaks with approximately correct molecular masses, there is still no assurance that proper disulfide bridges have been formed. Thus, to ensure the specificity of the anti-HIV-1 activity of commercial α-defensin preparations, virus inhibition assays were repeated with these peptides but now in the presence of an anti-α-defensin antibody. FIG. 4 (left panel) shows that the antibody indeed neutralized substantially the anti-HIV-1 activity of commercial peptides. This result suggests that the antiviral effect is not mediated by non-specific contaminants in the commercial preparations; instead, the activity resides in elements that are recognized by the anti-α-defensin antibody. The anti-HIV-1 activity of α-defensins-1, -2, and -3 purified from neutrophils of a normal person (36, 39) was also examined. This preparation contained α-defensin peaks that are virtually indistinguishable by mass spectrometry from those found in supernatants of CD8 T cells from LTNP-5 (FIG. 8). It too inhibited HIV-1 replication in vitro but with IC₅₀ of ˜0.5 to 2.2 μM (FIG. 4, right panel), suggesting that purified α-defensins are about 10-20 fold more potent against HIV-1 than commercial products. The antiviral effect of purified human neutrophil α-defensins was also substantially reduced or eliminated by the addition of the α-defensin-specific antibody

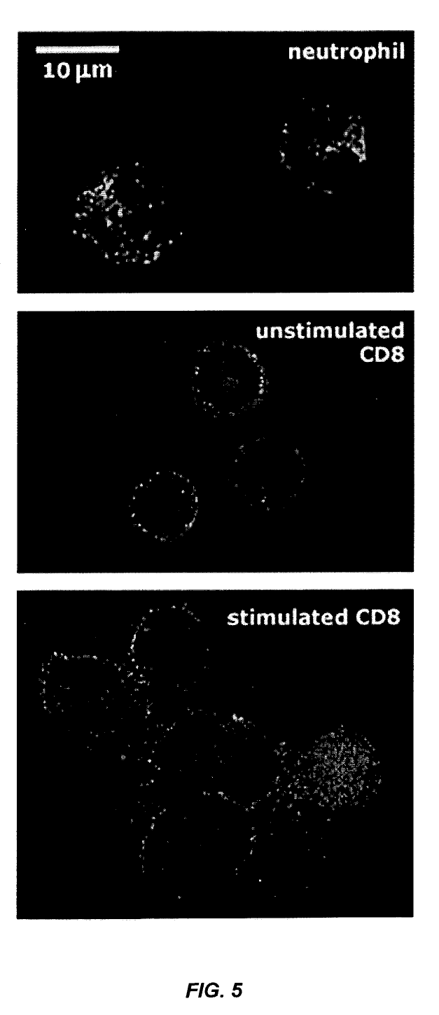

A subset of CD8 T lymphocytes express α-defensins-1, -2, and -3. Neutrophils and CD8 T cells purified from several normal blood donors were studied by immunofluorescence for intracellular expression of α-defensins-1, -2, and -3. A fraction of unstimulated CD8 T lymphocytes carried these molecules within small cytoplasmic granules, but in quantities considerably less than that found in neutrophils (FIG. 5). Upon stimulation, some of the CD8 T cells seemed to lose the α-defensin-positive granules, presumably due to secretion into the culture supernatant. A small percentage of CD8 T cells were activated to express a higher amount of α-defensins (FIG. 5; cell on the extreme right).

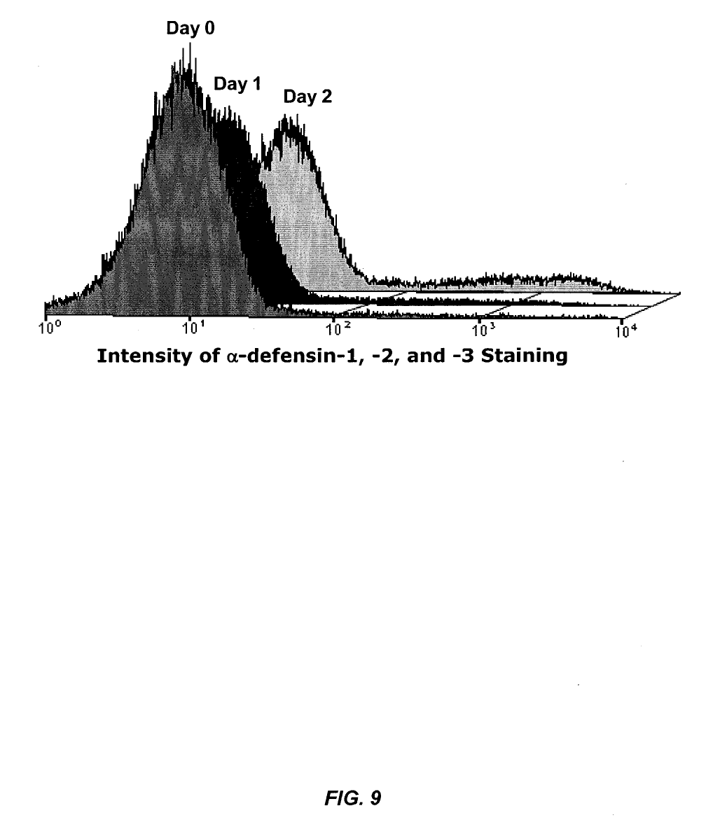

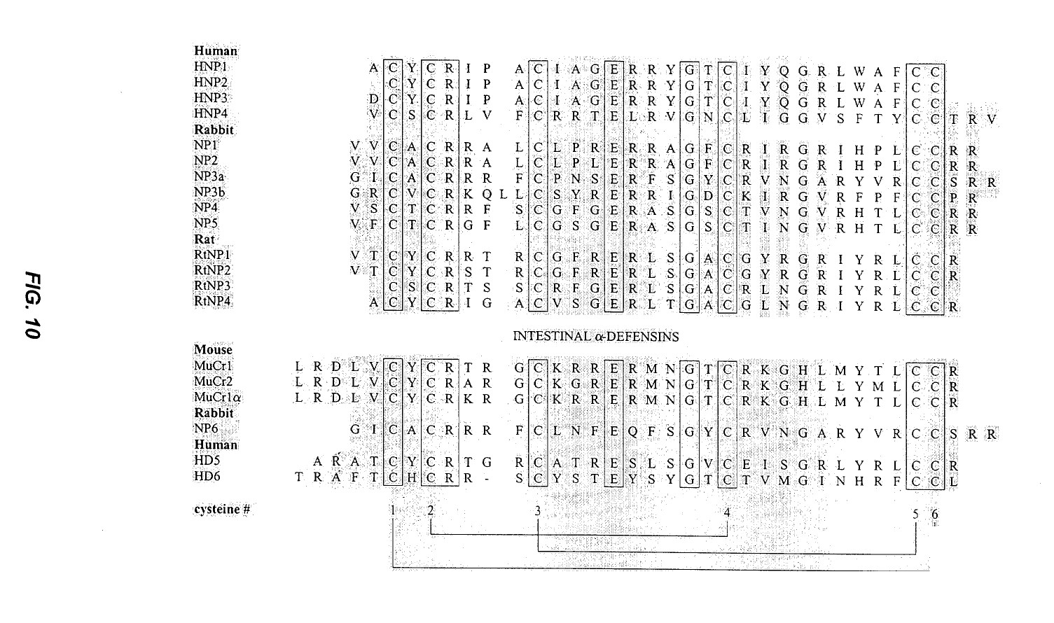

By flow cytometric analysis, about 2.3% of unstimulated αβ CD8 T lymphocytes expressed appreciable levels of α-defensins (FIG. 9). After one day of stimulation, some of the α-defensin-containing cells were no longer detectable. However, consistent with the immunofluorescence results, a sizeable population of CD8 T cells (21.1%) expressing higher amounts of α-defensins emerged after two days of stimulation. The α-defensin-positive CD8 cells were predominantly αβ T cells without γδ or NK markers. These findings further confirm that CD8 T cells do indeed harbor and secrete α-defensins-1, -2, and -3, establishing yet another linkage between innate and acquired immune systems.

### Example II

This example demonstrates that alpha-defensins levels in HIV-infected patient serum can serve as prognostic markers.

A study was undertaken to determine if the alpha-defensins varied in level of expression in the plasma of HIV-infected patients. Patient plasma was collected and assayed for defensins both by SELDI and by standard ELISA (HNP1-3 ELISA, Cell Sciences), and plasma from normal individuals (pooled, ordered from Sigma) was used as a positive control. From each of 21 patients, defensins were measured at two timepoints: just prior to the beginning of HAART therapy, and exactly one year after starting HAART therapy. All of the patients' viral loads were measureable at the beginning, and undetectable at the end of one year of HAART therapy. Their T cell counts (both CD4 and CD8) are know for both before and after HAART therapy. Table 2 summarizes the results.

The study found that while defensins were present in the uninfected control human plasma at concentrations of 6.2-7 ng/ml, out of the 21 HIV-infected individuals, the defensins could only be detected in 33% (7/21). Of the 7 patients in which defensins could be detected, three of them had low initial viral loads, suggesting the possibility that as viral load increases, defensin production is affected. 4 of the 7 patients that had detectable defensin levels in the beginning of the study lost expression partially or totally after one year of being on HAART therapy, despite the fact that their viral loads after one year were undetectable and their CD4 and CD8 counts were normal.

One patient had defensin levels that were undetectable at the beginning of the study, but after his CD4 count tripled during HAART therapy, defensins were detectable at about ⅓ the level of a normal individual. Two other patients had defensins present at low levels (again about ⅓ of the levels of an uninfected individual) both pre- and post-HAART therapy.

These results establish that in HIV-infected individuals, alpha-defensin levels are lower in the plasma than in uninfected individuals. This corroborates the in vitro finding that defensins stop viral replication, and are secreted by the CD8 T cells of normal controls and long term non-progressors (LTNP), but not by CD8 cells from AIDS patients.

TABLE 2

| | Pre-HAART avg | | Post-HAART avg | | | | |
|---|---|---|---|---|---|---|---|
| Patient | ng/ml | | ng/ml | | Initial Viral | CD4 | CD8 |
| # | defensins | | defensins | | Load | pre/post | pre/post |
| 14 | n/d | | n/d | | 128,122 | | |
| 28 | n/d | | n/d | | 2,257,800 | | |
| 38 | n/d | | n/d | | 81,176 | | |
| 6 | n/d | | n/d | | 93,386 | | |
| 37 | n/d | | n/d | | 593 | | |
| 22 | | 3.99 | | 0.47 | 10503 | 479/490 | 696/376 |
| 33 | | 6.52 | | 0 | 6681 | 572/558 | 1194/514 |
| 23 | | 2.84 | | 0 | 500 | 964/821 | 1799/1457 |
| 41 | | 6.72 | | 0 | 2,996,600 | 338/811 | 450/853 |
| 29 | n/d | | n/d | | 622,800 | | |
| 3 | n/d | | n/d | | 223,370 | | |
| 20 | | 2.22 | | 2.96 | 618,193 | 750/610 | 638/305 |
| 31 | | 2.01 | | 2.63 | 1,020,000 | 445/685 | 2104/318 |
| 4 | n/d | | n/d | | 266,895 | | |
| 12 | n/d | | n/d | | 71,791 | | |
| 32 | n/d | | n/d | | 7,553 | | |
| 21 | n/d | | n/d | | 571,216 | | |
| 16 | n/d | | n/d | | 112,593 | | |
| 5 | | 0 | | 2.24 | 50,880 | 390/812 | 858/464 |
| 27 | n/d | | n/d | | 100,000 | | |
| 9 | n/d | | n/d | | 141,902 | | |
| Control | | 6.2 | | | | | |
| 1 | | | | | | | |
| Control | | 7.08 | | | | | |
| 2 | | | | | | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A composition comprising a defensin polypeptide and a pharmaceutically acceptable carrier.

2. The composition of claim 1 wherein the defensin polypeptide is an alpha-defensin polypeptide.

3. The composition of claim 2 wherein the alpha-defensin polypeptide is selected from the group consisting of alpha-defensin 1, alpha-defensin 2 and alpha-defensin 3.

4. The composition of claim 1 comprising at least two alpha-defensin polypeptides.

5. The composition of claim 4 comprising alpha-defensin 1 and alpha-defensin 2.

6. The composition of claim 4 comprising alpha-defensin 1, alpha-defensin 2 and alpha-defensin 3.

7. A method of treating HIV infection in a human comprising administering a defensin polypeptide to the human.

8. The method of claim 7, further comprising administering a defensin polypeptide and a second therapeutic agent to the human.

9. The method of claim 7 wherein the defensin polypeptide is an alpha-defensin polypeptide selected from the group consisting of alpha-defensin 1, alpha-defensin 2 and alpha-defensin 3.

10. The method of claim 7 further comprising administering a second alpha-defensin polypeptide selected from the group consisting of alpha-defensin 1, alpha-defensin 2 and alpha-defensin 3.

11. The method of claim 9 wherein the alpha-defensin polypeptides are alpha-defensin 1 and alpha-defensin 2.

12. A method comprising administering a prophylactic amount of a defensin polypeptide to a person who is at high risk of HIV infection.

13. The method of claim 11 wherein the defensin polypeptide is an alpha-defensin polypeptide selected from the group consisting of alpha-defensin 1, alpha-defensin 2 and alpha-defensin 3.

14. The method of claim 11 further comprising administering a second alpha-defensin polypeptide selected from the group consisting of alpha-defensin 1, alpha-defensin 2 and alpha-defensin 3.

15. The method of claim 13 wherein the alpha-defensin polypeptides are alpha-defensin 1 and alpha-defensin 2.

16. A method of inhibiting HIV infection of a CD4⁺ cell in culture comprising the step of contacting the cell with a defensin polypeptide.

17. The method of claim 15 wherein the defensin polypeptide is an alpha-defensin polypeptide selected from the group consisting of alpha-defensin 1, alpha-defensin 2 and alpha-defensin 3.

18. The method of claim 15 further comprising administering a second alpha-defensin polypeptide selected from the group consisting of alpha-defensin 1, alpha-defensin 2 and alpha-defensin 3.

19. A composition comprising a first nucleic acid encoding a first defensin polypeptide in a pharmaceutically acceptable carrier.

20. The composition of claim 18 further comprising a second nucleic acid encoding a second defensin polypeptide.

21. The composition of claim 19 wherein the defensin polypeptide is an alpha-defensin polypeptide selected from the group consisting of alpha-defensin 1, alpha-defensin 2 and alpha-defensin 3.

22. The method of claim 20 wherein the alpha-defensin polypeptides are alpha-defensin 1 and alpha-defensin 2.

23. A method of inhibiting HIV infection in a human comprising transfecting a cell in vivo with a nucleic acid comprising a nucleotide sequence encoding a defensin polypeptide or transfecting a cell ex vivo with a nucleic acid comprising a nucleotide sequence encoding a defensin polypeptide and administering the cell to the human.

24. The method of claim 22 wherein the defensin polypeptide is an alpha-defensin polypeptide selected from the group consisting of alpha-defensin 1, alpha-defensin 2 and alpha-defensin 3.

25. The method of claim 22 wherein the cell is a muscle cell.

26. The method of claim 22 wherein the cell is a AC133⁺ or CD34⁺ progenitor cell.

27. The method of claim 22 wherein transfection is performed ex vivo.

28. The method of claim 22 wherein the nucleic acid comprises an inducible promoter operatively linked to the nucleotide sequence encoding the defensin polypeptide.

29. A method of determining an individual's HIV infection status comprising:
a) detecting an amount of alpha-defensin in a sample from the individual; and
b) correlating the amount with HIV infection status.

30. The method of claim 28 further comprising correlating the amount of defensin and at least one other indicator selected from the group consisting of CD8⁺ cell count, CD4⁺ cell count, HIV viral load and total T cell count with HIV infection status.

31. The method of claim 28 wherein the alpha-defensin is detected by ELISA or SELDI.

32. The method of claim 28 wherein the alpha-defensin polypeptide is selected from the group consisting of alpha-defensin 1, alpha-defensin 2 and alpha-defensin 3.

33. A method of determining an individual's HIV infection status comprising:
a) detecting an amount of alpha-defensin mRNA in a sample from the individual; and
b) correlating the amount with HIV infection status.

34. The method of claim 32 wherein the alpha-defensin polypeptide is selected from the group consisting of alpha-defensin 1, alpha-defensin 2 and alpha-defensin 3.

35. A method for determining whether a compound modulates alpha-defensin activity in a cell comprising:
a) contacting an alpha-defensin-producing cell with the compound; and
b) determining the functional effect of the compound on alpha-defensin activity.

36. The method of claim 34 wherein the cell is selected from the group consisting of a neutrophil, a CD8⁺ cell and an epithelial cell.

37. The method of claim 34 wherein the functional effect is determined by measuring defensin expression levels, cellular proliferation, or HIV replication.

38. The method of claim 34 wherein the compound is a small organic molecule.

39. The method of claim 34 wherein the alpha-defensin polypeptide is selected from the group consisting of alpha-defensin 1, alpha-defensin 2 and alpha-defensin 3.

40. A method for inhibiting HIV infection in a human comprising administering to the human a compound identified by the method of claim 35 to augment alpha-defensin activity.

41. A method comprising:
a) contacting a cell with alpha-defensin;
b) contacting an anti-alpha-defensin antibody with a lysate of the cell;
c) immobilizing the anti-alpha-defensin antibody to a solid phase before or after step (b), whereby alpha-defensin from the cell is bound to the solid phase; and
d) detecting proteins bound to the immobilized alpha-defensin.

42. A method comprising:
a) contacting a cell with alpha-defensin;
b) contacting an alpha-defensin with a lysate of the cell;
c) immobilizing the alpha-defensin to a solid phase before or after step (b), whereby proteins binding to alpha-defensin are captured; and
d) detecting proteins bound to the immobilized alpha-defensin.

43. The method of claim 40 or 41 wherein the cell is a CD4⁺ cell, a HeLa cell or a HOS cell.

44. The method of claim 40 or 41 wherein the alpha-defensin polypeptide is selected from the group consisting of alpha-defensin 1, alpha-defensin 2 and alpha-defensin 3.

45. A kit comprising (a) a substrate comprising means to bind an antibody; (b) an antibody against an alpha-defensin polypeptide; and (c) instructions to correlate an amount of an alpha-defensin detected in a patient sample with a prognosis of developing AIDS.

46. The kit of claim 44 wherein the substrate is a mass spectrometer probe.

47. The kit of claim 44 wherein the substrate is a microtiter plate.

48. The kit of claim 44 further comprising a second antibody specifically directed against alpha-defensin 1, alpha-defensin 2 or alpha-defensin 3.

49. The kit of claim 44 wherein the second antibody is labeled.

50. A kit comprising (a) a substrate comprising means to bind an alpha-defensin polypeptide; and (b) instructions to correlate an amount of an alpha-defensin detected in a patient sample with a prognosis of developing AIDS.

51. A kit comprising a first antibody that specifically binds an alpha-defensin and a second antibody that specifically binds alpha-defensin-1, alpha-defensin-2 or alpha-defensin-3.

52. An alpha-defensin fusion protein comprising a first alpha-defensin polypeptide moiety and a second moiety that binds CD4.

53. An alpha-defensin fusion protein comprising a first alpha-defensin polypeptide moiety and a second signal peptide moiety.

54. The alpha-defensin fusion protein of claim 53 wherien the second signal peptide moiety is a TPA signal peptide.

55. A nucleic acid comprising a nucleotide sequence encoding a polypeptide of claim 52.

56. An alpha-defensin fusion protein comprising a first alpha-defensin polypeptide moiety and a second moiety that increases stability or bioavailability of the protein in vivo.

57. A pharmaceutical composition comprising PEGylated alpha-defensin and a pharmaceutically acceptable carrier.

58. An alpha-defensin analog comprising amino acid substitutions that eliminate proteolytic cleavage sites.

59. A method of increasing endogenous alpha-defensin production comprising administering a composition that activates CD8⁺ cells.

60. The method of claim 59 wherein the composition comprises anti-CD3.

61. A method comprising:
a) expanding CD8⁺ cells ex vivo;
b) monitoring alpha-defensin production by the CD8⁺ cells; and
c) administer the expanded CD8⁺ cells an HLA-matched person.

62. A method comprising administering a vaccine and an alpha-defensin polypeptide or nucleic acid encoding alpha-defensin to a person.

63. A composition comprising a vaccine and an alpha-defensin polypeptide or nucleic acid encoding an alpha-defensin.

64. A composition comprising an alpha-defensin polypeptide and a second therapeutic agent.
